(19) United States
(12) Patent Application Publication
(10) Pub. No.: US 2010/0189681 A1
(43) Pub. Date: Jul. 29, 2010

(54) Methods of Using Phosphoantigens Together with Interleukin-2 for the Treatment of Cancer

(75) Inventor: Helene Sicard, Marseille (FR)

Correspondence Address: SALIWANCHIK LLOYD & SALIWANCHIK;A PROFESSIONAL ASSOCIATION, PO Box 142950, GAINESVILLE, FL, 32614, US

(73) Assignee: INNATE PHARMA S.A., Marseille (FR)

(21) Appl. No.: 12/601,628

(22) PCT Filed: May 21, 2008
(86) PCT No.: PCT/IB08/02197

§ 371 Date: Dec. 22, 2009

Related U.S. Application Data
(60) Provisional application No. 60/941,441, filed on Jun. 1, 2007.

(51) Int. Cl. A61K 38/20; A61P 35/00

(52) U.S. Cl. 424/85.2

(57) ABSTRACT

The present invention therefore provides novel approaches and strategies for efficient regulation of γδ T cells in vivo, in a subject, particularly a human subject or a non-human primate. The present invention now discloses particular compositions and methods that can be used to induce the proliferation of γδ T cells in vivo, in a subject. These compositions and methods employ the conjoint treatment of an individual with a γδ T cell activating compound and IL-2 and are particularly suited for immunotherapy in a subject, particularly in a subject having a cancer or an infectious disease.

### FIELD OF THE INVENTION

The present invention relates to compositions and methods for regulating an immune response in a subject, particularly a T cell response in a subject. The present invention more specifically discloses efficient methods of regulating the innate immunity in a subject, such as by regulating the activity of γδT cells in a subject. The invention discloses that particular combinations of particular agents, such as a cytokine and a γδT cell activator, can produce a remarkable expansion of γδT cells in vivo and a remarkable increase in a subject's immune defense. The invention can be used for therapeutic purposes, to produce, regulate or facilitate an immune response in a subject. It is particularly suited to regulate a protective immune response in subjects having a cancer or an infectious disease.

### BACKGROUND OF THE INVENTION

T cells of the γδ type are expressed by most mammalian species. They represent 1-10% of total circulating lymphocytes in healthy adult human subjects and most non-human primates. Most human peripheral blood γδ T cells express a γδTCR heterodimer encoded by Vγ9/Vδ2 genes, some NK-lineage receptors for MHC class I and almost no CD4 nor CD8. These cells have been shown to exhibit strong, non MHC-restricted, cytolytic activity against virus-infected cells (Poccia et al, J. Leukocyte Biology, 62, 1997, p. 1-5), parasite-infected cells (Constant et al, Infection and Immunity, vol. 63, no. 12, December 1995, p. 4628-4633), or tumor cells (Fournie et al, Bonneville, Res. Immunol., 66^{th} FORUM IN IMMUNOLOGY, 147, p. 338-347). These cells are also physiologically amplified in the context of several unrelated infectious diseases such as tuberculosis, malaria, tularemia, colibacillosis and also by B-cell tumors (for review see Hayday, 2000).

These cells are thus viewed as potent effectors of innate immunity. Because of their potent activity against tumor cells or infected cells, γδ T cells represent a very attractive candidate for immunotherapies. Notably, since activated γδ T cells exert potent cytolytic activity and T_{H}-1 cytokine secretion, these cells represent an important resource of anti-infectious and antitumoral effectors. Accordingly, it would be highly valuable to have methods available for regulating the activity (including the expansion and/or cytolytic activity, for instance), of these cells in vivo, in a subject.

In microbes, Vγ9/Vδ2⁺ lymphocytes spontaneously recognize a structurally related set of nonpeptide antigens, referred to as natural phosphoantigens and alkylamines. In B cell tumors, the nature of antigens for the γδ T cells remains unidentified. Vγ9/Vδ2⁺ lymphocytes are also responsive to a variety of virally infected-, activated- or tumoral cell types without prior exposure. Again, in these situations, the responsible antigens remain unknown (for review see Fisch, 2000). It has been shown that, in vitro, Vγ9/Vδ2⁺ lymphocytes respond to synthetic drugs such as therapeutic aminobisphosphonates (reviewed in Espinosa, 2001), leading to their in vitro activation. Recognition of natural non-peptide antigens is mediated by the γδ TCR, through amino acid residues located on both Vg9- and Vδ2- CDR3 regions. Although neither processing nor presentation by CD1 or MHC molecules is involved, Vγ9/Vδ2⁺ lymphocyte activation by non-peptide antigens appears to require cell-to-cell contact (Lang, 1995; Morita, 1995; Miyagawa, 2001; Rojas, 2002). The first set of clinical evidence for in vivo expansion of human Vγ9/Vδ2⁺ lymphocytes induced by phosphoantigen agonists came from the finding of increases of circulating γδ T cells within one to three weeks in humans adults with multiple myeloma after therapeutic intravenous injection of 60-90 mg of pamidronate (Kunzmann, 1999). However, the actual mode of action of pamidronate is unclear and might involve an indirect effect on accessory cells (Miyagawa, 2001) and no appropriate conditions were disclosed to allow optimized expansion of γδ T cells using such compound.

The inventors and/or their colleagues have previously characterized a number of immunomodulatory compounds capable of modulating the activity and proliferation of γδ T cells. These compounds (also called “phosphoantigens”) generally share a common structure in that they are organophosphate compounds. The classes having greatest potency are more particularly phosphate esters and phospho-phosphoroamidate esters. The particular compounds used by the inventors and their colleagues in a lead clinical trial comprise a pyrophosphate moiety. The lead molecule of this series of compounds developed by the inventors and their colleagues is called Phosphostim™, which is at present being evaluated in two Phase II clinical trials in oncology. A number of other compounds which activate γδ T cells are known as well, although most of these act indirectly (e.g. act on other immune cells which in turn activate γδ T cells) or act directly on γδ T cells but are less potent.

### Methods of Treatment Involving Administration of γδ T Cell Activating Compounds

In one aspect, research into treatment regimens based on γδ T cell activating compounds has been hampered by the lack of suitable in vivo models. Evidence for a general immune surveillance function of the innate immune system has been provided by various in vivo models: mice deficient in innate effector cells such as NK cells, NKT cells or γδ T cells show a significantly increased incidence of tumors (Girardi et al., 2001; Kim et al., 2000; Smyth et al., 2000). However, such results can only be transposed to the human situation with caution, as these cell populations are somewhat different in humans as compared to mice. In particular, the human Vγ9/Vδ2 cell population for example does not have a formal equivalent in rodents.

Furthermore, no therapeutic strategy involving stimulating a manifold increase of circulating γδ T cells in vivo had been developed, particularly for the treatment of tumors, including but not limited to solid tumors and those with metastases.

Previous work has highlighted the interest of the combination of γδ T cell activators and IL-2 to provide a γδ T cell expansion (Wilhelm et al, Blood, 2003, Sicard et al, J. Immunol. 2005, patent application EP 1 426 052, WO 04/050 096). Doses chosen were typically of about 1 MIU/m² daily.

IL-2 is known as a toxic product (grade III or IV toxicity have been reported), IL-2 treatments can induce severe side effect such as anemia, thrombocytopenia, anorexia, confusion, headaches, chills, hypotension, in some patients side effect have even been life threatening. The standard treatment dose, which is typically of about 18 MIU/m² daily, is aimed to activate all lymphocyte cell subtypes at the same time; this global activation induces many undesirable toxic side effects. A balance has to be determined for each patient between minimal effective dose of IL-2, to be administered with the γδ T cell activator, to trigger targeted γδ T cell expansion and tolerance towards IL-2. As the toxicity of IL-2 is dose dependant, one important point is to determine at which dose one can obtain the maximum therapeutic effect with as few side effects as possible.

Consequently, clinical trials combining various treatments have been set up. Formerly published work (Wilhelm et al, Blood, 2003) indicated that a very low dose of IL-2, around 1 MIU/m² daily, would be the dose of choice to trigger a specific γδ T cell expansion with acceptable toxicity. However, the γδ T cell expansion was very low (30 to 128 of increase in γδ T cells in percentage (0.3 to 1.28 fold), compared to a mean of 12 fold (1200%) in the present invention), presumably due to the nature γδ T cell activator (pamidronate) which typically produces only low levels of γδ T cell expansion. Such a low dose of IL-2 (1 MIU/m² daily per human patient) has been widely believed as the dose avoiding undesirable side effects and still causing an activation of certain cell subpopulations. Based on these documents and from experimental in vitro data and previous preliminary primate experiments (Sicard et al, J. Immunol. 2005), it was speculated that a low IL2 dose equivalent to 1 MIU/m² in human (equivalent to about 1.5 to 2.1 MIU/patient) was sufficient to trigger an efficient γδ T cell expansion.

In view of the foregoing, although several compounds have been shown to have in vitro and/or in vivo activity, their in vivo activity and more generally the in vivo kinetics of γδ T cells in response to stimulation had not been widely explored. Accordingly, efficient methods are needed to selectively activate γδ T cells in vivo, in a subject, under conditions suitable for therapy.

### SUMMARY

The present invention is based on observations during (i) studies in non-human primates and (ii) a human clinical trial using bromohydrin pyrophosphate (BrHPP, also referred to as Phosphostim™), where it was observed that this compound, when used in certain therapeutic regimens with interleukin-2 (IL-2) leads to an improved γδ T cell activation and proliferation, including a strong cytokine secretion and vigorous γδ T cell expansion in vivo. Finally, it was observed that higher rates of γδ T cell amplification can be achieved in vivo than had been previously known to be possible in humans and non-human primates, particularly in human patients having cancers.

The present invention therefore provides novel approaches and strategies for efficient regulation of γδ T cells in vivo, in a subject, particularly a human subject or a non-human primate.

The present invention now discloses particular compositions and methods that can be used to induce the proliferation of γδ T cells in vivo, in a subject. These compositions and methods employ the conjoint treatment of an individual with a γδ T cell activating compound and IL-2 and are particularly suited for immunotherapy in a subject, particularly in a subject having a cancer or an infectious disease.

The present invention provides a method of regulating the activity of γδ T cells, of preventing or treating a tumor, or for killing a tumor cell in a mammalian subject comprising conjointly administering to a subject an effective amount of a γδ T cell activator and an interleukin-2 polypeptide, the interleukin-2 polypeptide being administered at a dose of between 3.3 to 6 MIU/m² daily.

In another aspect, the invention provides a method of regulating the activity of γδ T cells, of preventing or treating a tumor, or for killing a tumor cell in a human subject comprising conjointly administering to a subject an effective amount of a γδ T cell activator and an interleukin-2 polypeptide, the interleukin-2 polypeptide being administered at a dose of between 7 and 9 MIU total daily.

In another aspect, the invention provides a method for inducing the proliferation of γδ T cells without inducing more than grade II toxicity in a mammalian subject comprising conjointly administering to a subject an effective amount of a γδ T cell activator and an interleukin-2 polypeptide. In one embodiment, in said method, the interleukin-2 polypeptide being administered at a dose of between 3.3 and 6 MIU/m² daily. In one embodiment, in said method, the interleukin-2 polypeptide being administered at a dose of between 7 and 9 MIU total daily in human.

In another embodiment, in the methods provided by the invention the γδ T cell activator is administered as a single dose on the first day of a treatment cycle with said γδ T cell activator. In another embodiment, in the methods provided by the invention the interleukin-2 is administered on at least two days during the first 10 days of said treatment cycle.

In an embodiment, interleukin-2 is administered on at least three days during the first 10 days of a treatment cycle. Preferably, interleukin-2 is administered on at least five days during the first 10 days of a treatment cycle.

In a further aspect, interleukin-2 is administered on at least 3 consecutive days of a treatment cycle. Preferably, interleukin-2 is administered on at least 5 consecutive days of a treatment cycle. In another aspect, interleukin-2 is administered on at least day 1 to day 3 of a treatment cycle. In another aspect, interleukin-2 is administered on at least day 1 to day 5 of a treatment cycle.

The present invention also provides a method of regulating the activity of γδ T cells, of preventing or treating a tumor, or for killing a tumor cell in a mammalian subject comprising:
a. administering to the subject an effective amount of a γδ T cell activator on a first day, and
b. administering to the subject an interleukin-2 polypeptide on at least 3 days within the days starting on the day of said administration of a γδ T activator, wherein the interleukin-2 is administered at a dose of between 3.3 to 6 MIU/m² daily or at a dose of between 7 and 9 MIU total daily in human.

The present invention also provides a method of regulating the activity of γδ T cells, of preventing or treating a tumor, or for killing a tumor cell in a mammalian subject comprising:
a. administering to the subject an effective amount of a γδ T cell activator on a first day, and
b. administering to the subject an interleukin-2 polypeptide on at least 5 consecutive days starting on the day of said administration of a γδ T cell activator, wherein the interleukin-2 is administered at a dose of between 3.3 to 6 MIU/m² daily or at a dose of between 7 and 9 MIU total daily in human.

The present invention also provides the use of an interleukin-2 polypeptide, for the manufacture of a pharmaceutical composition for regulating the activity of γδ T cells in a mammalian subject, the interleukin-2 polypeptide being administered at a dose of between 3.3 and 6 MIU/m² daily and in conjunction with a γδ T cell activator.

In another aspect, the invention relates to the use of an interleukin-2 polypeptide, for the manufacture of a pharmaceutical composition for regulating the activity of γδ T cells in a human subject, the interleukin-2 polypeptide being administered at a dose of between 7 and 9 MIU total daily and in conjunction with a γδ T cell activator.

In a further aspect, the present invention relates to the use of an interleukin-2 polypeptide, for the manufacture of a pharmaceutical composition, for use in conjunction with a γδ T cell activator for inducing the proliferation of γδ T cells without inducing more than grade II toxicity in a mammalian subject.

In an embodiment of the present invention, interleukin-2 polypeptide is being administered at a dose of between 3.3 and 6 MIU/m² daily, or at a dose of between 7 and 9 MIU total daily to a human.

In an embodiment, the γδ T cell activator is administered as a single dose on the first day of a treatment cycle with said γδ T cell activator. In other embodiments, interleukin-2 is administered on at least two days during the first 10 days of said treatment cycle, preferably on at least three days during the first 10 days of a treatment cycle, more preferably on at least five days during the first 10 days of a treatment cycle.

In another aspect, in the use provided by the present invention, said interleukin-2 is administered on at least 3 consecutive days of a treatment cycle, preferably, on at least 5 consecutive days of a treatment cycle.

In another embodiment, interleukin-2 is administered on at least day 1 to day 3 of a treatment cycle, preferably on at least day 1 to day 5 of a treatment cycle.

Preferably, in an aspect of the invention, interleukin-2 polypeptide is administered at a daily dose of about 8 MIU total in human. In a further embodiment, interleukin-2 polypeptide is aldesleukin. In an embodiment of the present invention, the γδ T cell activator is of the Formula I to III, more particularly the γδ T cell activator is of the Formula A, B, C, D E, F, G or H. In an embodiment, in the methods according to the invention, the dose of γδ T cell activator to be administered is comprised between 1 mg/kg to 100 mg/kg. In another aspect, the mammalian subject is a human subject suffering from a cancer or an infectious disease. In another aspect, the human subject suffers from a cancer selected from a solid tumor cancer or a hematopoietic cancer. In another aspect, the tumor to be treated is a solid tumor, a lymphoma or a leukemia, in particular a solid tumor, more preferably a renal carcinoma. In another aspect, the tumor to be treated is a non Hodgkin's lymphoma. In another aspect, the methods of the invention comprise conjointly administering a chemotherapeutic agent to said subject, more preferably antibodies, preferably rituximab, or tyrosine kinase inhibitors, preferably imatinib, sunitinib or sorafenib.

The present invention also provides a product comprising a γδ T cell activator and an interleukin-2 polypeptide, for separate use, for regulating the activity of γδ T cells in a mammalian subject, wherein the dose of IL-2 is between 3.3 and 6 MIU/m² daily. The present invention also provides a product comprising a γδ T cell activator and an interleukin-2 polypeptide, for separate use, for regulating the activity of γδ T cells in a human subject, wherein the dose of IL-2 is between 7 and 9 MIU total daily. The present invention also provides a product comprising a γδ T cell activator and an interleukin-2 polypeptide, for separate use, for regulating the activity of γδ T cells, without inducing more than grade II toxicity in a mammalian subject, the interleukin-2 polypeptide being administered in conjunction with a γδ T cell activator.

In an embodiment, interleukin-2 polypeptide is being administered at a dose of between 3.3 to 6 MIU/m² daily, or at a dose of between 7 and 9 MIU total daily in human, preferably at a daily dose of about 8 MIU total in human.

Preferably, in further aspects of the methods, uses and products provided by the invention, interleukin-2 polypeptide is administered at a daily dose of about 8 MIU total in human. In a further embodiment, interleukin-2 polypeptide is aldesleukin. In an embodiment of the present invention, the γδ T cell activator is of the Formula Ito III, more particularly the γδ T cell activator is of the Formula A, B, C, D E, F, G or H. In an embodiment, in the methods according to the invention, the dose of γδ T cell activator to be administered is comprised between 1 μg/kg to 100 mg/kg. In another aspect, the mammalian subject is a human subject suffering from a cancer or an infectious disease. In another aspect, the human subject suffers from a cancer selected from a solid tumor cancer, preferably mRCC or a hematopoietic cancer, preferably a non Hodgkin's lymphoma. In another aspect, the tumor to be treated is a solid tumor, a lymphoma or leukemia, in particular a solid tumor, more preferably a renal carcinoma. In another aspect, the tumor to be treated is a non Hodgkin's lymphoma. In another aspect, the methods of the invention comprise conjointly administering a chemotherapeutic agent to said subject, more preferably antibodies, preferably rituximab, or tyrosine kinase inhibitors, preferably imatinib, sunitinib or sorafenib.

Preferably the γδ T cell activator is selected from the group of: a compound capable of selectively activating a γδ T cell, a compound capable of activating a γδ T cell in a substantially pure culture of γδ T cells and a compound of Formulas I to III. In the framework of the present invention, the expression “Formulas I to III”, designate all compounds derived from Formulas I to III: I, II, IIa, III, IIIa, IIIa1, IIIa2, IIIa3, A, B, IIIb, IIIb1, IIIb2, IIIb3, C, IIIc, IIIc1, IIIc2, IIIc3, D, E, F, G and H. Most preferably, the compounds are selected from the list consisting of BrHPP, CBrHPP, EpoxPP, HDMAPP, C-HDMAPP, NHDMAPP, H-tiglylPP and H-angelylPP.

The inventors have found out that the combination of a γδ T cell activator and IL-2 at a dose, higher than what has been widely recommended, of about 4 MIU/m², could trigger an efficient γδ T cell proliferation with reduced side effects. The inventors have surprisingly found out that there exists a specific dose range effect on γδ T cell and that one can define an activation plateau for the combination of a γδ T cell activator and IL-2, achieving the maximum effect at a dose of 4 MIU/m² daily. The most efficient dose seems to be 1.2 MIU/animal daily (equivalent to 4 MIU/m² daily) whereas a further increase of IL-2 does not lead to an increased expansion. Indeed inventors have found out that a dose of IL-2 of about 4 MIU/m² daily is the lowest dose inducing the greatest γδ T cell activation and the most limited undesirable side effects. Moreover, as shown in FIG. 3, this dose does not lead to a substantial activation of NK cells nor of T cells globally. The invention thus provides a combination of a γδ T cell activator and IL-2 at a dose that is efficient for the induction of a specific expansion of γδ T cells, whereas other cell subsets remains substantially unaffected by the IL-2 administration.

The present invention thus relates to the use of a γδ T cell activator and an interleukin-2 polypeptide, for the manufacture of a pharmaceutical composition for regulating the activity of γδ T cells in a mammalian subject. The invention provides the use of an interleukin-2 polypeptide, for the manufacture of a pharmaceutical composition for regulating the activity of γδ T cells in a mammalian subject, wherein the IL-2 polypeptide or pharmaceutical composition is for conjoint use with a γδ T cell activator. The inventors have found out that the administration of a γδ T cell activator together with a particular dose of IL-2 enables an enhanced expansion of γδ T cells in vivo. Preferably, the interleukin-2 polypeptide is administered a dose of 8 MIU per patient, which is equivalent to 3.8 to 5.3 Million International Units per square meter per day, depending on the body surface of the patient to treat.

Preferably the dose of IL-2 to be administered is comprised between ably between 7 and 9 MIU total in human, most preferably 8 MIU total in human, typically over a period of time comprised between 1 and 10 days. More specifically, the γδ T cell activator and interleukin-2 polypeptide are administered separately. In a preferred embodiment, the γδ T cell activator is administered in a single dose, typically at the beginning of the treatment.

The invention also relates to the use of a γδ T cell activator and an interleukin-2 polypeptide, for the manufacture of a pharmaceutical composition for regulating the activity of γδ T cells in a mammalian subject, wherein the interleukin-2 polypeptide is administered at a dose between 3 and 5 MIU/m² per day, preferably 7 to 9 MIU total in human daily, even more preferably 8 MIU total in human daily. The γδ T cell activator is administered at a dose comprised between 1 μg/kg to 100 mg/kg. In an embodiment, a compound of Formula IIIa, especially a compound of Formula A or B is administered in a dosage (single administration) between about 1 mg/kg and about 100 mg/kg, preferably between about 10 mg/kg and about 80 mg/kg, more preferably between about 10 mg/kg and about 30 mg/kg. In another embodiment, a compound of Formula IIIc, especially, a compound of Formula D, E, F, G or H, is administered in a dosage (single administration) between about 1 μg/kg and about 100 mg/kg, preferably between about 10 μg/kg and about 20 mg/kg, more preferably between about 20 μg/kg and about 5 mg/kg, even more preferably between about 20 μg/kg and 2.5 mg/kg.

The invention also relates to a method of regulating the activity of γδ T cells in a mammalian subject, comprising separately administering to a subject in need thereof an effective amount of a γδ T cell activator and an interleukin-2 polypeptide. As indicated above, in a preferred embodiment, the interleukin-2 polypeptide is administered at a dose of about 8 MIU daily total in human, typically over a period of time comprised between 1 and 10 days, and/or the γδ T cell activator is administered in a single dose, typically at the beginning of the treatment.

Within the context of the present invention, the expression “regulating the activity of γδ T cells” designates causing or favoring an increase in the number and/or biological activity of such cells in a subject. Regulating thus includes modulating (e.g., stimulating) expansion of such cells in a subject and/or, for instance, triggering of cytokine secretion (e.g., TNFα or IFNγ). As indicated, γδ T cells normally represent between about 1-10% of total circulating lymphocytes in a healthy adult human subject. The present invention can be used to significantly increase the γδ T cells compartment in a subject, particularly to reach 30-90% of total circulating lymphocytes, typically 40-90%, more preferably from 50-90%. In typical embodiments, the invention allows the selective expansion of γδ T cells in a subject, to reach 60-90% of total circulating lymphocytes, preferably 70-90%, more preferably from 80-90%. Regulating also includes, in addition or in the alternative, modulating the biological activity of γδ T cells in a subject, particularly their cytolytic activity or their cytokine-secretion activity. The invention defines novel conditions and strategies for increasing the biological activity of γδ T cells towards target cells.

In the above methods and uses, the subject is preferably a human subject, such as a subject having a cancer, an infectious disease, an autoimmune disease or an allergic disease. The invention is indeed suitable to treat all conditions caused by or associated with the presence of pathological cells which are sensitive to γδ T cell lysis.

The invention is particularly suited to stimulate the anti-tumor immunity of a subject having a solid tumor, such as renal cell carcinoma, bladder cancer, breast cancer, colon cancer, multiple myeloma, etc.

In another embodiment, the invention is particularly suited to stimulate the anti-tumor immunity of a subject having a hematopoietic cancer, such as leukemia, non Hodgkin lymphoma.

The present invention more specifically relates to the use of a γδ T activator and an interleukin-2 polypeptide, for the manufacture of a pharmaceutical composition for treating cancer in a subject, wherein said γδ T cell activator and interleukin-2 polypeptide are administered separately to the subject. More preferably, the interleukin-2 polypeptide is administered at a dose of about 8 MIU total in human daily, typically over a period of time comprised between 1 and 10 days, and the γδ T cell activator is administered in a single dose at the beginning of the treatment.

The invention also relates to methods of treating a cancer in a subject, comprising separately administering to a subject in need thereof an effective amount of a γδ T cell activator and an interleukin-2 polypeptide. The dose of IL-2 being sufficient to induce an enhanced activation of γδT cells with a limited toxicity.

The above methods and treatments may be used alone or in combination with other active agents or treatments. For instance, for the treatment of tumors, the invention may be used in combination with other anti-tumor agents or treatments, such as antibody, tyrosine kinase inhibitors, chemotherapy, radiotherapy or gene therapy.

The invention also relates to a product comprising a γδ T cell activator and an interleukin-2 polypeptide, for separate use, for regulating the activity of γδ T cells in a mammalian subject, wherein the dose of IL-2 is of about 8 MIU total in human daily.

As indicated above, the invention is particularly suited for the treatment of tumors in a mammalian subject, such as a human subject.

### DESCRIPTION OF THE FIGURES

FIG. 1: IL-2 dose range effect on Vγ9δ2T cell rate with Phosphostim™ treatment on cynomolgus monkeys.

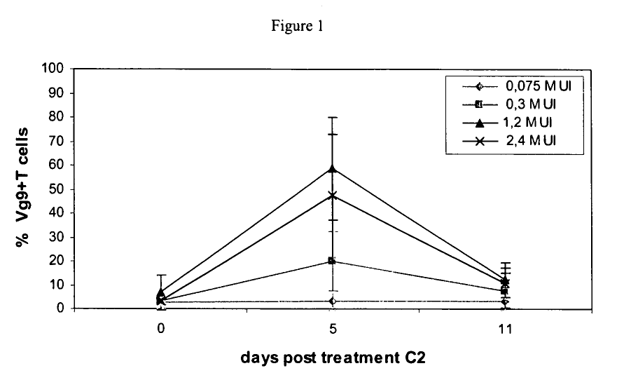

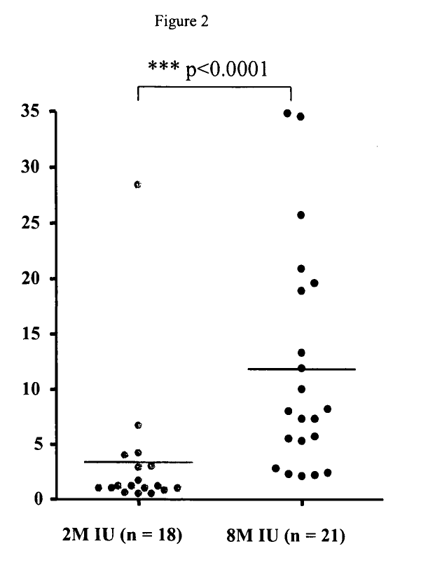

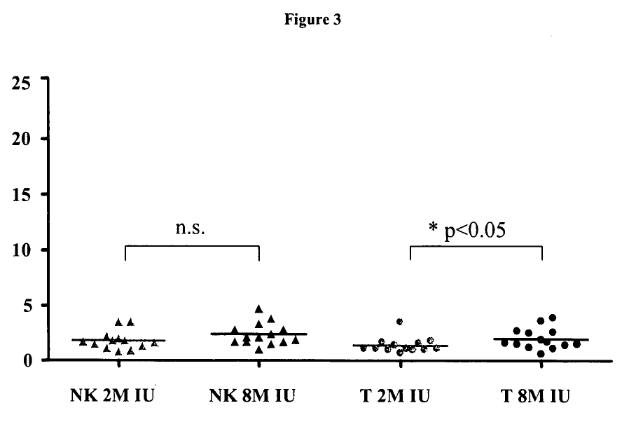

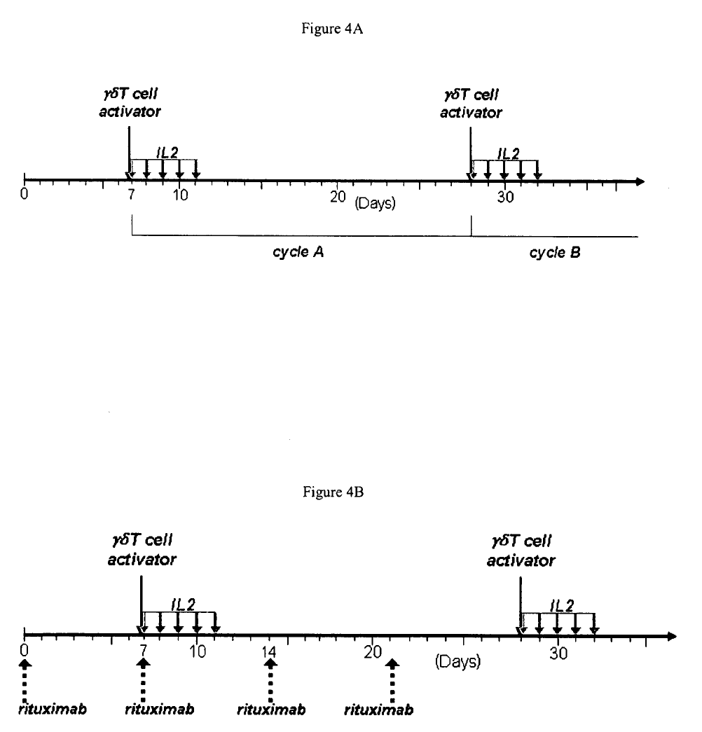

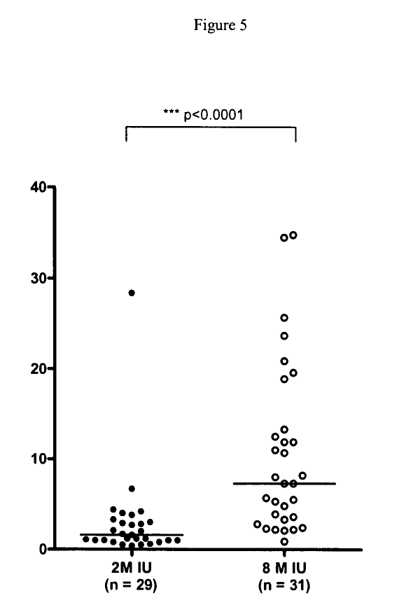

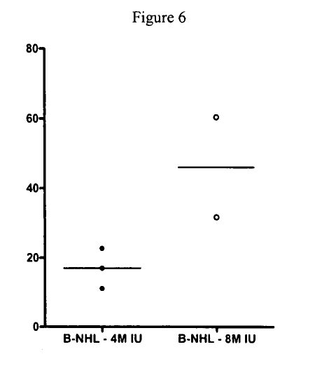

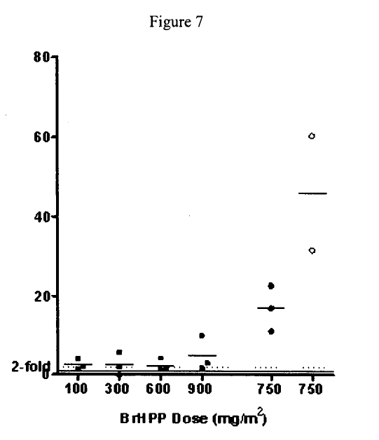

As detailed in Example 2, a study has been set up on nonhuman primates (Cynomolgus macaques) to investigate the effect of the IL-2 dose on γδ T cell expansion in vivo, with a fixed dose of Phosphostim™ (48 mg/kg). Blood extracts have been dosed by flow cytometry on day 5 and the γδ T cell expansion has been expressed in expansion percentage compared to the basal γδ T cell count. FIG. 1 shows the effect of various does of IL-2 on the γδ T cell expansion.

For the first doses, the expansion increases with the IL-2 dose, until 1.2 MIU/animal/daily, which corresponds to 4 MIU/m² daily, approximately 8 MIU total in human daily. The next highest dose, 2.4 MIU/animal/daily does not trigger a better expansion than 1.2 MIU/animal/daily, a plateau is observed at this dose which proves that a dose of about 4 MIU/m² is the most efficient dose.

FIG. 2: γδ T cell fold increase on day 7 as compared to pre-dose (measured on day 0).

FIG. 2 shows the effect of the IL-2 dose on γδ T cell subset. Patients suffering from mRCC are treated with 2 MIU total in human daily (arm A), or 8 MIU total in human daily (arm B).

Phosphostim™ is administered once on day 1 which is the first day of treatment. IL-2 is administered daily subcutaneous (s.c.) from day 1 to day 5. On day 1, IL-2 is administered 10 minutes after the end of Phosphostim™ infusion.

γδ T cell population is measured through cytometry on day 0 (before the beginning of the treatment) and on day 7. The expansion of γδ T cell is obtained in fold increase compared to the basal γδ T cell count.

The 21 patients treated with 8 MIU total in human daily (equivalent to approximately 4 MIU/m² daily) show a significantly better γδ T cell expansion (mean of 12 fold increase) than the 18 patients treated with 2 MIU total in human daily (mean of 3 fold increase).

All patients showed a good tolerance to the high dose of IL-2 administered (in Arm B), especially, there was no significant difference in toxicity in Arm B compared to Arm A.

FIG. 3: NK and total T cell fold increase on day 7 as compared to pre-dose (measured on day 0).

FIG. 3 has been obtained from observations made during the clinical trial as described above. On day 7, cell subsets were determined and compared to the count made on day 0, before the treatment. We do not observe a significant NK cells expansion, nor a significant total T cell expansion, which confirms that the IL-2 dose used is sub-therapeutic compared to a standard dose of 18 MIU/m² daily (that would induce an expansion of all T cell subsets).

These data confirm that the composition according to the invention enables a specific expansion of the γδ T cell population in cancer patients.

FIG. 4A: Preferred administration scheme for the γδ T cell activator and interleukin-2 polypeptide.

FIG. 4A describes a preferred embodiment for the administration of the active compounds to a patient.

On day 1, the γδ T cell activator is administered. Preferably in a short time after the γδ T cell activator administration, typically less than one hour after, the IL2 is administered. On following days (day 2, 3, 4, and 5), IL-2 is administered at the same dose than on day 1.

Then the patient is allowed to rest until the γδ T cell count has returned to basal rate, typically around day 21. This treatment scheme represents a treatment cycle A.

The administrations that give rise to a γδ T cell proliferation are separated by a period of time sufficient to prevent “exhaustion” of the γδ T cells. The second cycle A is administered preferably 3 weeks (around day 21) after the administration of the γδ T cell activator to the patient.

FIG. 4B: Preferred administration scheme for the γδ T cell activator, interleukin-2 polypeptide and monoclonal antibody (rituximab).

FIG. 4B describes a preferred embodiment for the administration of the active compounds to a patient.

On day 1, the monoclonal antibody is administered, the monoclonal antibody is administered 4 times, and each administration takes place 1 week apart. On day 7, the γδ T cell activator is administered. Preferably in a short time after the γδ T cell activator administration, typically less than one hour after, the IL2 is administered. On following days (day 8, 9, 10, and 11), IL-2 is administered at the same dose than on day 7.

Then the patient is allowed to rest until the γδ T cell count has returned to basal rate, typically around day 28. This treatment scheme represents a treatment cycle A.

The administrations that give rise to a γδ T cell proliferation are separated by a period of time sufficient to prevent “exhaustion” of the γδ T cells. The second cycle A is administered preferably 3 weeks (around day 28) after the administration of the γδ T cell activator to the patient.

FIG. 5: γδ T cell fold increase on day 7 as compared to pre-dose (measured on day 0).

FIG. 5 is the updated representation of the results obtained showed in FIG. 2. Results are obtained as described for FIG. 2. The effect of the IL-2 dose on γδ T cell subset in patients suffering from mRCC and treated with 2 different IL-2 doses: 2 MIU total in human daily (arm A), or 8 MIU total in human daily (arm B) is confirmed.

The 31 patients treated with 8 MIU total in human daily (equivalent to approximately 4 MIU/m² daily) show a significantly better γδ T cell expansion (mean of 8 fold increase) than the 29 patients treated with 2 MIU total in human daily (mean of 2 fold increase).

All patients showed a good tolerance to the high dose of IL-2 administered (in Arm B), especially, there was no significant difference in toxicity in Arm B compared to Arm A.

FIG. 6: γδ T cell fold increase on day 7 as compared to pre-dose (measured on day 0) in NHL patients treated with rituximab in combination.

FIG. 6 shows the effect of the IL-2 dose on γδ T cell subset on the first results obtained in another clinical trial. 5 patients suffering from B-NHL are treated with 4 MIU total in human daily (arm A, 3 patients), or 8 MIU total in human daily (arm B, 2 patients). The treatment regimen used for this study is set out in FIG. 4B.

Rituxan™ (rituximab) is administered at a dose of 375 mg/m², on day 0 and then on day 7, 14 and 21, which corresponds to the common Rituxan™ treatment. Phosphostim™ is administered once on day 7 which is the first day of treatment. IL-2 is administered daily subcutaneous (s.c.) from day 7 to day 11. On day 7, IL-2 is administered 10 minutes after the end of Phosphostim™ infusion.

γδ T cell population is measured through cytometry on day 0 (before the beginning of the treatment) and on day 145 (corresponds to day 7 from the beginning of the Phosphostim™ treatment. The expansion of γδ T cell is obtained in fold increase compared to the basal γδ T cell count.

The 2 patients treated with 8 MIU total in human daily (equivalent to approximately 4 MIU/m² daily) show a significantly better γδ T cell expansion (mean of 46 fold increase) than the 3 patients treated with 4 MIU total in human daily (mean of 17 fold increase).

All patients showed a good tolerance to the high dose of IL-2 administered (in Arm B), especially, there was no significant difference in toxicity in Arm B compared to Arm A.

FIG. 7: γδ T cell fold increase on day 7 as compared to pre-dose (measured on day 0) in NHL patients treated or not with rituximab in combination.

FIG. 7 shows the effect of the IL-2 dose on γδ T cell subsets in phase I and phase II clinical trials. The first four columns (corresponding to 100, 300, 600 and 900 MU/m² doses of BrHPP) correspond to patients treated with BrHPP (iv, 30 min infusion on day 1) and IL-2 (1 MU/m² daily for four days starting on day 1) whereas the last two columns represent B-NHL patients who have been treated with 4 MIU total in human daily (arm A), or 8 MIU total in human daily (arm B), in the treatment setting described for FIG. 6.

The results highlight that the patients treated with a higher dose of IL-2 (8 MIU total daily) shows a much better γδ T cell amplification than patients treated with a lower dose of IL-2. The graph also shows that the quantity of BrHPP only as a slight influence on the γδ T cell amplification compared to the effect of the IL-2 dose. The present invention thus also extends to patients having a leukemia type cancer.

### DETAILED DESCRIPTION

### Definitions

Where “comprising” is used, this can preferably be replaced by “consisting essentially of”, more preferably by “consisting of”.

As used in the specification, “a” or “an” may mean one or more. As used in the claim(s), when used in conjunction with the word “comprising”, the words “a” or “an” may mean one or more than one. As used herein “another” may mean at least a second or more.

Where hereinbefore and hereinafter numerical terms are used, they are meant to include the numbers representing the upper and lower limits. For example, “between 1 and 3” stands for a range “from and including 1 up to and including 3”, and “in the range from 1 to 3” would stand for “from and including 1 up to and including 3”. The same is true where instead of numbers (e.g. 3) words denoting numbers are used (e.g. “three”).

“Weekly” stands for “about once a week” (meaning that more than one treatment is made with an interval of about one week between treatments), the about here preferably meaning +/−1 day (that is, translating into “every 6 to 8 days”); most preferably, “weekly” stands for “once every 7 days”.

“3-weekly” or “three-weekly” stands for “about once every three weeks” (meaning that more than one treatment is made with an interval of about three weeks between treatments), the about here preferably meaning +/−3 days (that is, translating into every 18 to 24 days); most preferably, “weekly” stands for “once every 21 days” (=every third week).

The term “about” or “approximately” usually means within 20%, more preferably within 10%, and most preferably still within 5% of a given value or range. Alternatively, especially in biological systems (e.g., when measuring an immune response), the term “about” means within about a log (i.e., an order of magnitude) preferably within a factor of two of a given value.

The term “limited toxicity” designates a toxicity that is under the level of detection of major side effects. A limited toxicity is typically a toxicity of grade I or II, such as defined by WHO (World Health Organization) Toxicity Criteria. Limited toxicity can induce acceptable side effects, i.e. not leading to a major patient discomfort, such a moderate fever (less than 40° C.) or chills for example.

Within the context of the present invention, the expressions “stimulating the activity of γδ T cells”, “activating γδ T cells” and “regulating the activity of γδ T cells” designate causing or favoring an increase in the number and/or biological activity of such cells in a subject. Stimulating and regulating thus each include without limitation modulating (e.g., stimulating) expansion of such cells in a subject and/or, for instance, triggering of cytokine secretion (e.g., TNFα or IFNγ). γδ T cells normally represent between about 1-10% of total circulating lymphocytes in a healthy adult human subject. The present invention can be used to significantly increase the γδ T cells population in a subject, particularly to reach at least 30% of total circulating lymphocytes, typically 40%, more preferably at least 50% or 60%, or from 50% to 90%. Regulating also includes, in addition or in the alternative, modulating the biological activity of γδ T cells in a subject, particularly their cytolytic activity or their cytokine-secretion activity. The invention defines novel conditions and strategies for increasing the biological activity of γδ T cells towards target cells.

In accordance with the present invention there may be employed conventional molecular biology, microbiology, and recombinant DNA techniques within the skill of the art. Such techniques are well-known and are explained fully in the literature. See, e.g., Sambrook, Fritsch and Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

As used herein, the terms “conjoint”, “in combination” or “combination therapy”, used interchangeably, refer to the situation where two or more therapeutic agents affect the treatment or prevention of the same disease. The use of the terms “conjoint”, “in combination” or “combination therapy” does not restrict the order in which therapies (e.g., prophylactic or therapeutic agents) are administered to a subject with the disease. A first therapy can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject with a disease.

Within the context of the present invention, the term “separately administered” indicates that the active ingredients are administered at a different site or through a different route or through a different schedule to the subject. Accordingly, the ingredients are generally not mixed together prior to administration, although they may be combined in a unique package in suitable separated containers.

### Cancer and Other Proliferative Diseases

A variety of cancers and other proliferative diseases including, but not limited to, the following can be treated using the methods and compositions of the invention:
carcinoma, including that of the bladder, breast, colon (e.g. colorectal cancer), kidney (e.g. renal cell cancer, mRCC), liver, lung (e.g. non-small cell lung cancer), ovary, pancreas, stomach, cervix, thyroid, urethra, fallopian tube, pelvis, prostate, testicules, peritoneal cavity, oesophage, gastrointestinal apparatus (e.g. GIST) and skin, including squamous cell carcinoma;
tumors of mesenchymal origin, including fibrosarcoma and rhabdomyoscarcoma;
other tumors, including melanoma, seminoma, teratocarcinoma, neuroblastoma and glioma, recurrent glioblastoma multiforme (GBM),
tumors of the central and peripheral nervous system, including astrocytoma, neuroblastoma, glioma, and schwannomas, Brain and Central Nervous System Tumors, Head and Neck Cancer, Cervical Cancer, Malignant Peripheral Nerve Sheath Tumors,
tumors of mesenchymal origin, including fibrosarcoma, rhabdomyoscaroma, and osteosarcoma; and
other tumors, including melanoma, xeroderma pigmentosum, keratoacarcinoma, seminoma, thyroid follicular cancer and teratocarcinoma.
leukemias such as, but not limited to, acute leukemia, acute lymphocytic leukaemia (ALL), acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukaemia (CML), chronic lymphocytic leukemia, hairy cell leukemia; polycythemia vera, chronic eosinophilic leukemia (CEL), Hypereosinophilic Syndrome.
lymphomas such as, but not limited to, Hodgkin's disease, non-Hodgkin's disease; multiple myelomas such as, but not limited to, smoldering multiple myeloma, nonsecretory myeloma, osteosclerotic myeloma, plasma cell leukemia, solitary plasmacytoma and extramedullary plasmacytoma.

Where hereinbefore and subsequently a tumor, a tumor disease, a carcinoma or a cancer are mentioned, also metastasis in the original organ or tissue and/or in any other location are implied alternatively or in addition, whatever the location of the tumor and/or metastasis is.

In an embodiment, the treatment according to the invention is particularly suited for the treatment of cancers, more preferably solid cancers, including but not limited to metastatic renal cell carcinoma (mRCC), renal cell carcinoma.

In an embodiment, the treatment according to the invention is particularly suited for the treatment of lymphoma, more preferably non Hodgkin's lymphoma.

In an embodiment, the treatment according to the invention is particularly suited for the treatment of leukemia, more preferably chronic myelocytic leukemia (CML).

### γδ T Cell Activators

The term “γδT cell activator” designates a molecule, preferably artificially produced, which can activate γδ T lymphocytes. It is more preferably a ligand of the T receptor of γδ T lymphocytes. The γδ T cell activators may by of various natures, such as a peptide, lipid, small molecule, etc. It may be a purified or otherwise artificially produced (e.g., by chemical synthesis, or by microbiological process) endogenous ligand, or a fragment or derivative thereof, or an antibody having substantially the same antigenic specificity.

A phosphoantigen that is a γδ T cell activator preferably increases the biological activity or causes the proliferation of γδ T cells, preferably increasing the activation of γδ T cells, particularly increasing cytokine secretion from γδ T cells or increasing the cytolytic activity of γδ T cells, with or without also stimulating the proliferation or expansion of γδ T cells. Accordingly, the γδ T cell activator is administered in an amount and under conditions sufficient to increase the activity γδ T cells in a subject, preferably in an amount and under conditions sufficient to increase cytokine secretion by γδ T cells and/or to increase the cytolytic activity of γδ T cells. Cytokine secretion and cytolytic activity can be assessed using any appropriate in vitro assay (as described hereunder).

Method for detecting activation of a γδ T cell will be carried out according to standard methods. For example, cytokine secretion and cytolytic activity can be assessed using any appropriate in vitro assay, or those provided in the examples herein. For example, cytokine secretion can be determined according to the methods described in Espinosa et al. (J. Biol. Chem., 2001, Vol. 276, Issue 21, 18337-18344), describing measurement of TNF-α release in a bioassay using TNFα sensitive cells. Briefly, 10⁴ γδ T cells/well were incubated with stimulus plus 25 units of IL2/well in 100 μl of culture medium during 24 h at 37° C. Then, 50 μl of supernatant were added to 50 μl of WEHI cells plated at 3×10⁴ cells/well in culture medium plus actinomycin D (2 μg/ml) and LiCl (40 mM) and incubated for 20 h at 37° C. Viability of the TNF-α-sensitive cells and measured with a 3-(4,5-dimethylthiazol-2-yl)-2,5-diphenyltetrazolium bromide assay. 50 μl of 3-(4,5-dimethylthiazol-2-yl)-2,5-diphenyltetrazolium bromide (Sigma; 2.5 mg/ml in phosphate-buffered saline) per well were added, and after 4 h of incubation at 37° C., 50 μl of solubilization buffer (20% SDS, 66% dimethyl formamide, pH 4.7) were added, and absorbance (570 nm) was measured. Levels of TNF-α release were then calculated from a standard curve obtained using purified human rTNF-α (PeproTech, Inc., Rocky Hill, N.J.). Interferon-γ released by activated T cells was measured by a sandwich enzyme-linked immunosorbent assay. 5×10⁴ γδ T cells/well were incubated with stimulus plus 25 units of IL2/well in 100 μl of culture medium during 24 h at 37° C. Then, 50 μl of supernatant were harvested for enzyme-linked immunosorbent assay using mouse monoclonal antibodies (BIOSOURCE, Camarillo, Calif.).

A preferred assay for cytolytic activity is a ⁵¹Cr release assay. In exemplary assays, the cytolytic activity of γδ T cells is measured against autologous normal and tumor target cell lines, or control sensitive target cell lines such as Daudi and control resistant target cell line such as Raji in 4 h ⁵¹Cr release assay. In a specific example, target cells were used in amounts of 2×10³ cells/well and labeled with 100 μCi ⁵¹Cr for 60 minutes. Effector/Target (E/T) ratio ranged from 30:1 to 3.75:1. Specific lysis (expressed as percentage) is calculated using the standard Formula

[(experimental−spontaneous release/total−spontaneous release)×100].

The term phosphoantigen designates a T lymphocyte agonist, more particularly a Tγδ lymphocyte agonist, whose activity depends on the presence of a phosphate moiety. It has been formerly described (see Espinosa et al, Microbes and Infections 2001, or Belmant et al, Drug discovery today 2005) that such compounds have a unique specificity to trigger a 78 T cell response.

As discussed, the methods of the invention can generally be carried out with any γδ T cell activator that is capable of stimulating γδ T cell activity. This stimulation can be by direct effect on γδ T cells as discussed below using compounds that can stimulate γδ T cells in a pure γδ T cell culture, or the stimulation can be by an indirect mechanism, such as treatment with pharmacological agents such as bisphosphonates which lead to IPP accumulation. Preferably, a γδ T cell activator is a compound capable of regulating the activity of a γδ T cells in a population of γδ T cell clones in culture. The γδ T cell activator is capable of regulating the activity of a γδ T cell population of γδ T cell clones at millimolar concentration, preferably when the γδ cell activator is present in culture at a concentration of less than 100 mM. Optionally a γδ T cell activator is capable of regulating the activity of a γδ T cell in a population of γδ T cell clones at millimolar concentration, preferably when the γδ T cell activator is present in culture at a concentration of less than 10 mM, or more preferably less than 1 mM. Regulating the activity of a γδ T cell can be assessed by any suitable means, preferably by assessing cytokine secretion, most preferably TNF-α secretion as described herein. Methods for obtaining a population of pure γδ T cell clones is described in Davodeau et al, (1993) and Moreau et al, (1986), the disclosures of which are incorporated herein by reference. Preferably the γδ T cell activator is capable of causing at least a 20%, 50% or greater increase in the number of γδ T cells in culture, or more preferably at least a 2-fold increase in the number of γδ T cells in culture.

In one embodiment, the γδ T cell activator may be a chemical compound capable of selectively activating Vγ9Vδ2 T lymphocytes. Selective activation of Vγ9Vδ2 T lymphocytes indicates that the compound has a selective action towards specific cell populations, preferably increasing activation of Vγ9Vδ2 T cells at a greater rate or to a greater degree than other T cell types such as Vγ1 T cells, or not substantially not activation other T cell types. Such selectivity can be assessed in vitro T cell activation assays. Such selectivity, as disclosed in the present application, suggests that preferred compounds can cause a selective or targeted activation of the proliferation or biological activity of Vγ9Vδ2 T lymphocytes.

Detection of γδ T cell proliferation can be detected by standard methods. One specific method for detecting γδ T cell amplification in vivo is described in Example 1.

### Preferred Phosphoantigens

In a preferred aspect, the γδ T cell activator may increase the biological activity of a γδ T cell, preferably increasing the activation of a γδ T cell, particularly increasing cytokine secretion from a γδ T cell or increasing the cytolytic activity of a cytotoxic γδ T cell, and/or stimulating the proliferation of a γδ T cell. Preferred γδ T cell activators include a composition comprising a compound of the Formula I, especially a γδ T cell activator according to Formulas Ito III, especially γδ T cell activator selected from the group consisting of BrHPP, CBrHPP, EpoxPP, HDMAPP, CHDMAPP, NHDMAPP, H-angelylPP and H-tiglylPP. In the framework of the present invention, the expression “Formulas I to III”, designate all compounds derived from Formulas Ito III: I, II, IIa, III, IIIa, IIIa1, IIIa2, IIIa3, A, B, Mb, IIIb2, IIIb3, C, IIIc, IIIc1, IIIc2, IIIc3, D, E, F, G and H. Most preferably, the γδ T cell activator is selected from the list consisting of BrIAPP, CBrHPP, EpoxPP, HDMAPP, CHDMAPP, NHDMAPP, H-angelylPP and H-tiglylPP. However, it will appreciated that a number of phosphoantigen compounds that are less potent γδ T cell activators are available and may be used in accordance with the invention. For example, in one variant, a bisphosphonate compounds such as pamidronate (Novartis, Nuernberg, Germany) or zoledronate may be used. Other γδ T cell activators for use in the present invention are phosphoantigens disclosed in WO 95/20673, isopentenyl pyrophosphate (IPP) (U.S. Pat. No. 5,639,653), the disclosures of the two preceding documents being incorporated herein by reference, as well as alkylamines (such as ethylamine, iso-propyulamine, n-propylamine, n-butylamine and iso-butylamine, for instance). Isobutyl amine and 3-aminopropyl phosphonic acid are obtained from Aldrich (Chicago, Ill.).

The γδ T cell activator is administered in an amount and under conditions sufficient to increase the activity γδ T cells in a subject, preferably in an amount and under conditions sufficient to increase cytokine secretion by γδ T cells and/or to increase the cytolytic activity of γδ T cells. In typical embodiments, a γδ T cell activator allows the cytokine secretion by γδ T cells to be increased at least 2, 3, 4, 10, 50, 100-fold, as determined in vitro.

In one aspect, a phosphoantigen according to the present invention comprises a compound of Formula (I):

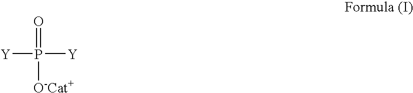

wherein:
Cat+ represents one (or several, identical or different) organic or mineral cation(s) (including proton);
Y represents O⁻Cat⁺, a C₁-C₆, or more preferably C₁-C₃, alkyl group, a group -A-R, a group -A-PO(O⁻Cat⁺)—Y, or a radical selected from the group consisting of a nucleoside, an oligonucleotide, a nucleic acid, an amino acid, a peptide, a protein, a monosaccharide, an oligosaccharide, a polysaccharide, a fatty acid, a simple lipid, a complex lipid, a folic acid, a tetrahydrofolic acid, a phosphoric acid, an inositol, a vitamin, a co-enzyme, a flavonoid, an aldehyde, an epoxyde and a halohydrin;
A represents O, NH, CHF, CF₂ or CH₂ or R₁—C—R₂ wherein R₁ and R₂ are selected from the group consisting of R, a hydrogen, an hydroxyl (—OH), and an halogen, but may vary independently of each other, respectively; and,
R is a linear, branched, or cyclic, aromatic or not, saturated or unsaturated, C₁-C₂₀ hydrocarbon group, optionally interrupted by at least one heteroatom, wherein said hydrocarbon group comprises an alkyl, an alkylenyl, or an alkynyl, preferably an alkyl or an alkylene, which can be substituted by one or several substituents selected from the group consisting of: an alkyl, an alkylenyl, an alkynyl, an epoxyalkyl, an aryl, an heterocycle, an alkoxy, an acyl, an alcohol, a carboxylic group (—COOH), an ester, an amine, an amino group (—NH₂), an amide (—CONH₂), an imine, a nitrile, an hydroxyl (—OH), a aldehyde group (—CHO), an halogen, an halogenoalkyl, a thiol (—SH), a thioalkyl, a sulfone, a sulfoxide, and a combination thereof.
Preferably, R₁ and R₂ are defined as any of the R₁ and R₂ shown in Table 1.

In another aspect, a phosphoantigen according to the present invention comprises a compound of Formula (II):

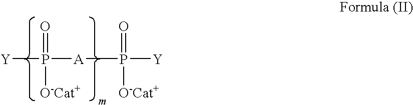

wherein:
Cat⁺ represents one (or several, identical or different) organic or mineral cation(s) (including proton);
m is an integer from 1 to 3;
Y represents O⁻Cat⁺, a C₁-C₆, or more preferably C₁-C₃, alkyl group, a group -A-R, or a radical selected from the group consisting of a nucleoside, an oligonucleotide, a nucleic acid, an amino acid, a peptide, a protein, a monosaccharide, an oligosaccharide, a polysaccharide, a fatty acid, a simple lipid, a complex lipid, a folic acid, a tetrahydrofolic acid, a phosphoric acid, an inositol, a vitamin, a co-enzyme, a flavonoid, an aldehyde, an epoxyde and a halohydrin;
R and A have the aforementioned meaning.

The term “independently” is used in the present specification to indicate that the variable which is independently applied varies independently from application to application. Thus, in a compound such as R—C—R, wherein “R varies independently as carbon or nitrogen”, both R can be carbon, either R can be nitrogen, or one R can be carbon and the other R nitrogen.

In further embodiments it will be appreciated that the present invention and particularly method for making crystalline phases is suitable for use with structurally related compounds to the ones mentioned specifically herein. In preferred embodiments, the invention also encompasses nucleotides and nucleotide analogs or derivatives or nucleotide-like compounds as well as a bisphosphonate compounds. In a preferred aspect, a compound of Formula II is a bisphosphonate compound, preferably a compound of Formula IIa. A bisphosphonate compound preferably comprises a structure of the Formula (IIa):

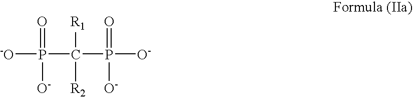

Preferably, R₁ and R₂ are defined as any of the R₁ and R₂ shown in Table 1

TABLE 1

| Agent | R₁ side chain | R₂ side chain |
|---|---|---|
| Etidronate | —OH | —CH₃ |
| Clodronate | —Cl | —Cl |
| Tiludronate | —H | 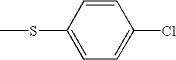 |
| Pamidronate | —OH | —CH₂—CH₂—NH₂ |
| Neridronate | —OH | —(CH₂)₅—NH₂ |
| Olpadronate | —OH | —(CH₂)₂—N(CH₃)₂ |
| Alendronate | —OH | —(CH₂)₃—NH₂ |
| Ibandronate | —OH | 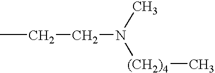 |
| Risedronate | —OH | 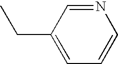 |
| Zoledronate | —OH | 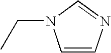 |

Preferably, a compound of the bisphosphonate type is selected from the group consisting of the following compounds or a pharmaceutically acceptable salt thereof, or any hydrate thereof: 3-amino-1-hydroxypropane-1,1-diphosphonic acid (pamidronic acid), e.g. pamidronate (APD); 3-(N,N-dimethylamino)-1-hydroxypropane-1,1-diphosphonic acid, e.g. dimethyl-APD; 4-amino-1-hydroxybutane-1,1-diphosphonic acid (alendronic acid), e.g. alendronate; 1-hydroxy-ethidene-bisphosphonic acid, e.g. etidronate; 1-hydroxy-3-(methylpentylamino)-propylidene-bisphosphonic acid, ibandronic acid, e.g. ibandronate; 6-amino-1-hydroxyhexane-1,1-diphosphonic acid, e.g. amino-hexyl-BP; 3-(N-methyl-N-pentylamino)-1-hydroxypropane-1,1-diphosphonic acid, e.g. methyl-pentyl-APD (=BM 21.0955); 1-hydroxy-2-(imidazol-1-yl)ethane-1,1-diphosphonic acid; 1-hydroxy-2-(3-pyridinyl)ethane-1,1-diphosphonic acid (risedronic acid), e.g. risedronate, including N-methyl pyridinium salts thereof, for example N-methylpyridinium iodides such as NE-10244 or NE-10446, 1-(4-chlorophenylthio)methane-1,1-diphosphonic acid (tiludronic acid), e.g. tiludronate; 3[N-(2-phenylthioethyl)-N-methylamino]-1-hydroxypropane-1,1-diphosphonic acid; 1-hydroxy-3-(pyrrolidin-1-yl)propane-1,1-diphosphonic acid, e.g. EB 1053 (Leo); 1-(N-phenylaminothiocarbonyl)methane-1,1-diphosphonic acid, e.g. FR 78844 (Fujisawa); 5-benzoyl-3,4-dihydro-2H-pyrazole-3,3-diphosphonic acid tetraethyl ester, e.g. U-81581 (Upjohn); 1-hydroxy-2-(imidazo[1,2-a]pyridin-3-yl)ethane-1,1-diphosphonic acid, e.g. YM 529; and 1,1-dichloromethane-1,1-diphosphonic acid (clodronic acid), e.g. clodronate. Preferably the bisphosphonate are compounds which lead to activation of γδ T cells. Examples of commercialized bisphosphonates are shown in Table 1 above, including the identity of R₁ and R₂ for each molecule.

Further examples of preferred phosphoantigens for use according to the present invention comprise the compounds of Formula (III):

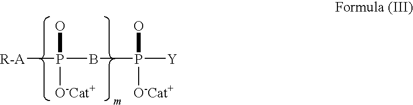

wherein:
Cat⁺ represents one (or several, identical or different) organic or mineral cation(s) (including proton);
m is an integer from 1 to 3;
B is O, NH, or any group capable to be hydrolyzed;
Y represents O⁻Cat⁺, a C₁-C₃ alkyl group, a group -A-R, or a radical selected from the group consisting of a nucleoside, an oligonucleotide, a nucleic acid, an amino acid, a peptide, a protein, a monosaccharide, an oligosaccharide, a polysaccharide, a fatty acid, a simple lipid, a complex lipid, a folic acid, a tetrahydrofolic acid, a phosphoric acid, an inositol, a vitamin, a co-enzyme, a flavonoid, an aldehyde, an epoxyde and a halohydrin;
A is O, NH, CHF, CF₂ or CH₂; and,
R is a linear, branched, or cyclic, aromatic or not, saturated or unsaturated, C₁-C₂₀ hydrocarbon group, optionally interrupted by at least one heteroatom, wherein said hydrocarbon group comprises an alkyl, an alkylenyl, or an alkynyl, preferably an alkyl or an alkylene, which can be substituted by one or several substituents selected from the group consisting of: an alkyl, an alkylenyl, an alkynyl, an epoxyalkyl, an aryl, an heterocycle, an alkoxy, an acyl, an alcohol, a carboxylic group (—COOH), an ester, an amine, an amino group (—NH₂), an amide (—CONH₂), an imine, a nitrile, an hydroxyl (—OH), a aldehyde group (—CHO), an halogen, an halogenoalkyl, a thiol (—SH), a thioalkyl, a sulfone, a sulfoxide, and a combination thereof. Most preferably, said phosphoantigen compound are γδ T cell activators.

In a particular embodiment of any of the Formulas disclosed for use in accordance with the invention, the substituents as defined above are substituted by at least one of the substituents as specified above.

Preferably, the substituents are selected from the group consisting of: an (C₁-C₆)alkyl, an (C₂-C₆)alkylenyl, an (C₂-C₆)alkynyl, an (C₂-C₆)epoxyalkyl, an aryl, an heterocycle, an (C₁-C₆)alkoxy, an (C₂-C₆)acyl, an (C₁-C₆)alcohol, a carboxylic group (—COOH), an (C₂-C₆)ester, an (C₁-C₆)amine, an amino group (—NH₂), an amide (—CONH₂), an (C₁-C₆)imine, a nitrile, an hydroxyl (—OH), an aldehyde group (—CHO), an halogen, an (C₁-C₆)halogenoalkyl, a thiol (—SH), a (C₁-C₆)thioalkyl, a (C₁-C₆)sulfone, a (C₁-C₆)sulfoxide, and a combination thereof.

More preferably, the substituents are selected from the group consisting of: an (C₁-C₆)alkyl, an (C₂-C₆)epoxyalkyl, an (C₂-C₆)alkylenyl, an (C₁-C₆)alkoxy, an (C₂-C₆)acyl, an (C₁-C₆)alcohol, an (C₂-C₆)ester, an (C₁-C₆)amine, an (C₁-C₆)imine, an hydroxyl, a aldehyde group, an halogen, an (C₁-C₆)halogenoallyl and a combination thereof.

Still more preferably, the substituents are selected from the group consisting of: an (C₃-C₆)epoxyallyl, an (C₁-C₃)alkoxy, an (C₂-C₃)acyl, an (C₁-C₃)alcohol, an (C₂-C₃)ester, an (C₁-C₃)amine, an (C₁-C₃)imine, an hydroxyl, an halogen, an (C₁-C₃)halogenoallyl, and a combination thereof. Preferably, R is a (C₃-C₂₅)hydrocarbon group, more preferably a (C₅-C₁₀)hydrocarbon group.

In the context of the present invention, the term “alkyl” more specifically means a group such as methyl, ethyl, propyl, isopropyl, butyl, isobutyl, tert-butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, heneicosyl, docosyl and the other isomeric forms thereof. (C₁-C₆)alkyl more specifically means methyl, ethyl, propyl, isopropyl, butyl, isobutyl, tert-butyl, pentyl, hexyl and the other isomeric forms thereof. (C₁-C₃)alkyl more specifically means methyl, ethyl, propyl, or isopropyl.

The term “alkenyl” refers to an alkyl group defined hereinabove having at least one unsaturated ethylene bond and the term “alkynyl” refers to an alkyl group defined hereinabove having at least one unsaturated acetylene bond. (C₂-C₆)alkylene includes a ethenyl, a propenyl (1-propenyl or 2-propenyl), a 1- or 2-methylpropenyl, a butenyl (1-butenyl, 2-butenyl, or 3-butenyl), a methylbutenyl, a 2-ethylpropenyl, a pentenyl (1-pentenyl, 2-pentenyl, 3-pentenyl, 4-pentenyl), an hexenyl (1-hexenyl, 2-hexenyl, 3-hexenyl, 4-hexenyl, 5-hexenyl), and the other isomeric forms thereof. (C₂-C₆)alkynyl includes ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl and the other isomeric forms thereof.

The term “epoxyalkyl” refers to an alkyl group defined hereinabove having an epoxide group. More particularly, (C₂-C₆)epoxyalkyl includes epoxyethyl, epoxypropyl, epoxybutyl, epoxypentyl, epoxyhexyl and the other isomeric forms thereof. (C₂-C₃)epoxyalkyl includes epoxyethyl and epoxypropyl.

The “aryl” groups are mono-, bi- or tri-cyclic aromatic hydrocarbons having from 6 to 18 carbon atoms. Examples include a phenyl, α-naphthyl, β-naphthyl or anthracenyl group, in particular.

“Heterocycle” groups are groups containing 5 to 8 rings comprising one or more heteroatoms, preferably 1 to 5 endocyclic heteroatoms. They may be mono-, bi- or tri-cyclic. They may be aromatic or not. Preferably, and more specifically for R₅, they are aromatic heterocycles. Examples of aromatic heterocycles include pyridine, pyridazine, pyrimidine, pyrazine, furan, thiophene, pyrrole, oxazole, thiazole, isothiazole, imidazole, pyrazole, oxadiazole, triazole, thiadiazole and triazine groups. Examples of bicycles include in particular quinoline, isoquinoline and quinazoline groups (for two 6-membered rings) and indole, benzimidazole, benzoxazole, benzothiazole and indazole (for a 6-membered ring and a 5-membered ring). Nonaromatic heterocycles comprise in particular piperazine, piperidine, etc.

“Alkoxy” groups correspond to the alkyl groups defined hereinabove bonded to the molecule by an —O— (ether) bond. (C₁-C₆)alkoxy includes methoxy, ethoxy, propyloxy, butyloxy, pentyloxy, hexyloxy and the other isomeric forms thereof. (C₁-C₃)alkoxy includes methoxy, ethoxy, propyloxy, and isopropyloxy.

“Alkyl” groups correspond to the alkyl groups defined hereinabove bonded to the molecule by an —CO— (carbonyl) group. (C₂-C₆)acyl includes acetyl, propylacyl, butylacyl, pentylacyl, hexylacyl and the other isomeric forms thereof. (C₂-C₃)acyl includes acetyl, propylacyl and isopropylacyl.

“Alcohol” groups correspond to the alkyl groups defined hereinabove containing at least one hydroxyl group. Alcohol can be primary, secondary or tertiary. (C₁-C₆)alcohol includes methanol, ethanol, propanol, butanol, pentanol, hexanol and the other isomeric forms thereof. (C₁-C₃)alcohol includes methanol, ethanol, propanol and isopropanol.

“Ester” groups correspond to the alkyl groups defined hereinabove bonded to the molecule by an —COO— (ester) bond. (C₂-C₆)ester includes methylester, ethylester, propylester, butylester, pentylester and the other isomeric forms thereof. (C₂-C₃)ester includes methylester and ethylester.

“Amine” groups correspond to the alkyl groups defined hereinabove bonded to the molecule by an —N-(amine) bond. (C₁-C₆)amine includes methylamine, ethylamine, propylamine, butylamine, pentylamine, hexylamine and the other isomeric forms thereof. (C₁-C₃)amine includes methylamine, ethylamine, and propylamine.

“Imine” groups correspond to the alkyl groups defined hereinabove having a (—C═N—) bond. (C₁-C₆)imine includes methylimine, ethylimine, propylimine, butylimine, pentylimine, hexylimine and the other isomeric forms thereof. (C₁-C₃)imine includes methylimine, ethylimine, and propylimine.

The halogen can be Cl, Br, I, or F, more preferably Br or F.

“Halogenoalkyl” groups correspond to the alkyl groups defined hereinabove having at least one halogen. The groups can be monohalogenated or polyhalogenated containing the same or different halogen atoms. For example, the group can be a trifluoroalkyl (CF₃—R). (C₁-C₆)halogenoallyl includes halogenomethyl, halogenoethyl, halogenopropyl, halogenobutyl, halogenopentyl, halogenohexyl and the other isomeric forms thereof. (C₁-C₃)halogenoalkyl includes halogenomethyl, halogenoethyl, and halogenopropyl.

“Thioalkyl” groups correspond to the alkyl groups defined hereinabove bonded to the molecule by an —S-(thioether) bond. (C₁-C₆)thioalkyl includes thiomethyl, thioethyl, thiopropyl, thiobutyl, thiopentyl, thiohexyl and the other isomeric forms thereof. (C₁-C₃)thioalkyl includes thiomethyl, thioethyl, and thiopropyl.

“Sulfone” groups correspond to the alkyl groups defined hereinabove bonded to the molecule by an —SOO— (sulfone) bond. (C₁-C₆)sulfone includes methylsulfone, ethylsulfone, propylsulfone, butylsulfone, pentylsulfone, hexylsulfone and the other isomeric forms thereof. (C₁-C₃)sulfone includes methylsulfone, ethylsulfone and propylsulfone.

“Sulfoxyde” groups correspond to the alkyl groups defined hereinabove bonded to the molecule by an —SO— (sulfoxide) group. (C₁-C₆)sulfoxide includes methylsulfoxide, ethylsulfoxide, propylsulfoxide, butylsulfoxide, pentylsulfoxide, hexylsulfoxide and the other isomeric forms thereof. (C₁-C₃)sulfoxide includes methylsulfoxide, ethylsulfoxide, propylsulfoxide and isopropylsulfoxide.

“Heteroatom” denotes N, S, or O.

“Nucleoside” refers to a compound composed of any pentose or modified pentose moiety attached to a specific position of a heterocycle or to the natural positions of a purine (9-position) or pyrimidine (1-position) or to the equivalent position in an analog. The term nucleoside includes but is not limited to adenosine, thymine, uridine, cytidine and guanosine.

In a particular embodiment, the hydrocarbon group is a cycloalkylenyl such as a cyclopentadiene or a phenyl, or an heterocycle such as a furan, a pyrrole, a thiophene, a thiazole, an imidazole, a triazole, a pyridine, a pyrimidine, a pyrane, or a pyrazine. Preferably, the cycloalkylenyl or the heterocycle is selected from the group consisting of a cyclopentadiene, a pyrrole or an imidazole. In a preferred embodiment, the cycloalkylenyl or the heterocycle is substituted by an alcohol. Preferably, said alcohol is a (C₁-C₃)alcohol.

In another embodiment, the hydrocarbon group is an alkylenyl with one or several double bonds. Preferably, the alkylenyl group has one double bond. Preferably, the alkylenyl group is a (C₃-C₁₀)alkylenyl group, more preferably a (C₄-C₇)alkylenyl group. Preferably, said alkylenyl group is substituted by at least one functional group. More preferably, the functional group is selected from the group consisting of an hydroxy, an (C₁-C₃)alkoxy, an aldehyde, an (C₂-C₃)acyl, or an (C₂-C₃)ester. In a more preferred embodiment, the hydrocarbon group is butenyl substituted by a group —CH₂OH. Optionally, said alkenyl group can be the isoform trans (E) or cis (Z), more preferably a trans isoform (E). In a most preferred embodiment, the alkylenyl group is the (E)-4-hydroxy-3-methyl-2-butenyl. In an other preferred embodiment, the alkylenyl group is an isopentenyl, a dimethylallyl or an hydroxydimethylallyl.

In an additional embodiment, the hydrocarbon group is an alkyl group substituted by an acyl. More preferably, the hydrocarbon group is an (C₄-C₇)alkyl group substituted by an (C₁-C₃)acyl.

In a further preferred embodiment, the phosphoantigen is of Formula (IIIc):

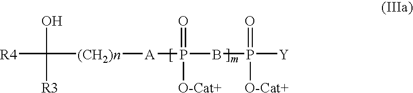

in which:
R₄ is an halogenated (C₁-C₃)alkyl, a (C₁-C₃)alkoxy-(C₁-C₃)allyl, an halogenated (C₂-C₃)acyl or a (C₁-C₃)alkoxy-(C₂-C₃)acyl,
R₃ is (C₁-C₃)alkyl group,
m is an integer from 1 to 3,
n is an integer from 2 to 20,
B represents O, NH, or any group capable to be hydrolyzed
A represents O, NH, CHF, CF₂ or CH₂,
Y represents O⁻Cat⁺, a C₁-C₃ alkyl group, a group -A-R, or a radical selected from the group consisting of a nucleoside, an oligonucleotide, a nucleic acid, an amino acid, a peptide, a protein, a monosaccharide, an oligosaccharide, a polysaccharide, a fatty acid, a simple lipid, a complex lipid, a folic acid, a tetrahydrofolic acid, a phosphoric acid, an inositol, a vitamin, a co-enzyme, a flavonoid, an aldehyde, an epoxyde and a halohydrin;
R is a linear, branched, or cyclic, aromatic or not, saturated or unsaturated, C₁-C₅₀ hydrocarbon group, optionally interrupted by at least one heteroatom, wherein said hydrocarbon group comprises an alkyl, an alkylenyl, or an alkynyl, preferably an alkyl or an alkylene, which can be substituted by one or several substituents selected from the group consisting of: an alkyl, an alkylenyl, an alkynyl, an epoxyalkyl, an aryl, an heterocycle, an alkoxy, an acyl, an alcohol, a carboxylic group (—COOH), an ester, an amine, an amino group (—NH₂), an amide (—CONH₂), an imine, a nitrile, an hydroxyl (—OH), a aldehyde group (—CHO), an halogen, an halogenoalkyl, a thiol (—SH), a thioalkyl, a sulfone, a sulfoxide, and a combination thereof. Most preferably, said phosphoantigen compound are γδ T cell activators, and
Cat⁺ represents one (or several, identical or different) organic or mineral cation(s) (including the proton).

Preferably, R₄ is an halogenated methyl (—CH₂—X, X being an halogen), an halogenated (C₂-C₃)acetyl, or (C₁-C₃)alkoxy-acetyl. The halogenated methyl or acetyl can be mono-, di-, or tri-halogenated. More preferably, R₄ is a CH₂—X group, X represents a halogen atom. Preferably, X is selected from I, Br and Cl. Preferably, R₃ is a methyl or ethyl group. More preferably, R₃ is a methyl. Preferably, B is O and A is O or CH₂. Preferably, n is an integer from 2 to 10, or from 2 to 5. In a more preferred embodiment, n is 2. Preferably, m is 1 or 2. More preferably, m is 1. Preferably, Y is O⁻Cat⁺, or a nucleoside. More preferably, Y is O⁻Cat⁺.

In a most preferred embodiment, n is 2, R₃ is a methyl and R₄ is a halogenated methyl, more preferably a monohalogenated methyl, still more preferably a bromide methyl. In a particularly preferred embodiment, n is 2, R₃ is a methyl, R₄ is a methyl bromide. In a most preferred embodiment, R is 3-(bromomethyl)-3-butanol-1-yl.

In another embodiment, R₄ is a CH₂—X group and A and B represent O.

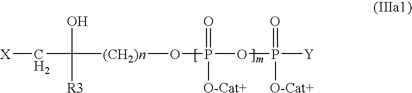

wherein R₃, X, n, m, Y and Cat⁺ have the aforementioned meaning. Preferably, R₃ is a methyl. Preferably, n is 2. Preferably, X is a bromide.

In another embodiment R₄ is a CH₂—X group and B represents O and A represents CH₂.

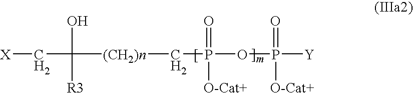

wherein R₃, X, n, m, Y and Cat⁺ have the aforementioned meaning.

Preferably, R₃ is a methyl. Preferably, n is 2. Preferably, X is a bromide.

In one preferred embodiment, a phosphoantigen comprises a compound of Formula (IIIa3):

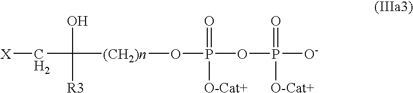

in which X is an halogen (preferably selected from I, Br and Cl), R₃ is a methyl or ethyl group, Cat⁺ represents one (or several, identical or different) organic or mineral cation(s) (including the proton), and n is an integer from 2 to 20. Preferably, R₃ is a methyl. Preferably, n is 2. Preferably, X is a bromide.

In a most preferred embodiment, a phosphoantigen comprises a compound of Formula (A):

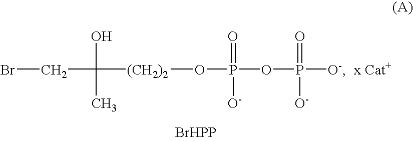

Preferably x Cat⁺ is 1 or 2 Na⁺.

In another most preferred embodiment, a phosphoantigen comprises a compound of Formula (B):

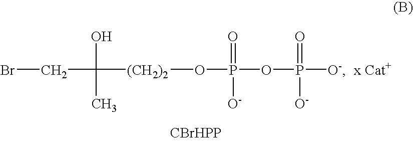

Preferably x Cat⁺ is 1 or 2 Na⁺.

In a further preferred embodiment, the phosphoantigen is of Formula (Mb):

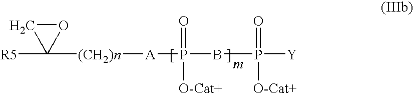

wherein:
n is an integer from 2 to 20,
m is an integer from 1 to 3,
R₅ is a methyl or ethyl group,
B represents O, NH, or any group capable to be hydrolyzed,
A represents O, NH, CHF, CF₂ or CH₂,
Y is O⁻Cat⁺, a nucleoside, or a radical -A-R, wherein R has the aforementioned meaning, and
Cat⁺ represents one (or several, identical or different) organic or mineral cation(s) (including the proton).

Preferably, n is an integer from 2 to 10, or from 2 to 5. In a more preferred embodiment, n is 2. Preferably, R₅ is a methyl. Preferably, Y is O⁻Cat % or a nucleoside. More preferably, Y is O-Cat⁺.

Preferably, A is O, NH or CH₂. More preferably, B is O. Preferably, B is O. Preferably, m is 1 or 2. More preferably, m is 1.

For example, a phosphoantigen may comprise a compound of Formula (IMO or (IIIb2):

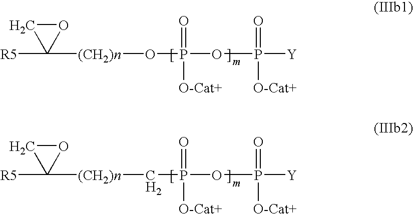

wherein R₅, n, m, Y and Cat⁺ have the above mentioned meaning.

In another preferred embodiment, a phosphoantigen comprises a compound of Formula (IIIb3):

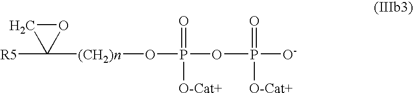

in which R₅ is a methyl or ethyl group, Cat⁺ represents one (or several, identical or different) organic or mineral cation(s) (including the proton), and n is an integer from 2 to 20. Preferably, R₅ is a methyl. Preferably, n is 2.

In another preferred embodiment a phosphoantigen comprises a compound of Formula (C):

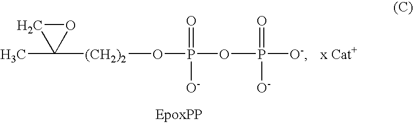

Preferably x Cat+ is 1 or 2 Na⁺.

In a further preferred embodiment, the phosphoantigen is of Formula (IIIc):

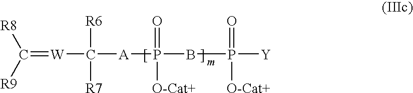

wherein:
R₆, R₇, and R₈ represent, independently from each other, a hydrogen atom or a (C₁-C₃)alkyl group,
R₉ is an (C₂-C₃)acyl, an aldehyde, an (C₁-C₃)alcohol, or an (C₂-C₃)ester,
W is —CH—, —N— or —C—R₁₀,
A is O, NH, CHF, CF₂ or CH₂,
B represents O, NH, or any group capable to be hydrolyzed,
m is an integer from 1 to 3,
Y is O⁻Cat⁺, a nucleoside, or a radical -A-R, wherein R has the aforementioned meaning.
Cat⁺ represents one (or several, identical or different) organic or mineral cation(s) (including the proton),

More preferably, R₆ and R₈ are a methyl and R₇ is hydrogen. More preferably, R₉ is —CHO, —CO—CH₃ or —CO—OCH₃. Optionally, the double-bond between W and C is in conformation trans (E) or cis (Z). More preferably, the double-bond between W and C is in conformation trans (E).

The group Y can permit the design of a prodrug. Therefore, Y is enzymolabile group which can be cleaved in particular regions of the subject. The group Y can also be targeting group. In a preferred embodiment, Y is O⁻Car, a group —B—R, or a radical selected from the group consisting of a nucleoside, a monosaccharide, an epoxyde and a halohydrin. Preferably, Y is an enzymolabile group.

Preferably, Y is O⁻Car, a group —B—R, or a nucleoside. In a first preferred embodiment, Y is O⁻Car. In a second preferred embodiment, Y is a nucleoside.

In a preferred embodiment, Cat⁺ is H⁺, Na⁺, NH₄⁺, K⁺, Li⁺, (CH₃CH₂)₃NH⁺.

In a preferred embodiment, B is O or NH. More preferably, B is O.

In a preferred embodiment, A is O, NH or CH₂.

In a preferred embodiment, m is 1 or 2. More preferably, m is 1.

In another example, a phosphoantigen comprises a compound of Formula (IIIc1) or (IIIc2):

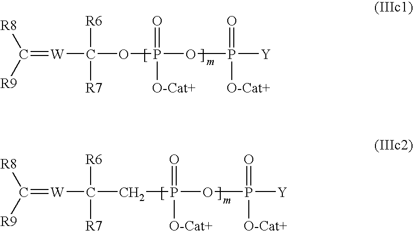

wherein R₆, R₇, R₈, R₉, R₁₀, W, m, Y and Cat⁺ have the above mentioned meaning.

In a preferred embodiment, W is —CH—. Preferably, R₆ and R₇ are hydrogen. Preferably, R₈ is a methyl. Preferably, R₉ is —CH₂—OH.

In another preferred embodiment, a phosphoantigen comprises a compound of Formula (D):

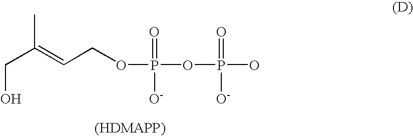

In yet another preferred embodiment, a phosphoantigen comprises a compound of Formula (E):

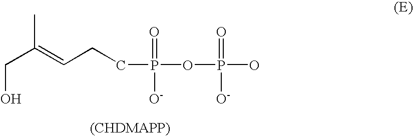

In yet another preferred embodiment, a phosphoantigen comprises a compound of Formula (F):

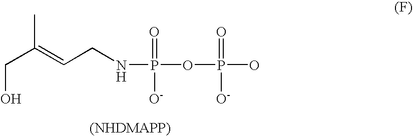

In another example, phosphoantigen comprises a compound of Formula (IIIc3):

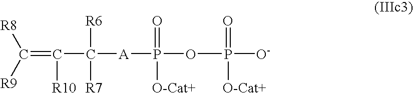

wherein R₆, R₇, R₈, R₉, R₁₀, and A have the above mentioned meaning.

Preferably, R₆, R₇ and R₉ are hydrogen. Preferably, R₁₀ is a methyl. Preferably, R₈ is —CH₂—OH. Preferably, A is CH₂, NH or O.

In a preferred embodiment, a phosphoantigen comprises a compound named (Z)-5-hydroxy-3-methylpent-3-enyl pyrophosphonate, of Formula G:

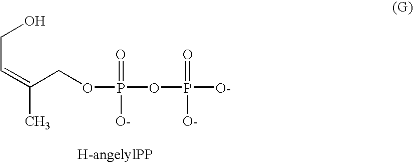

In further embodiments, the γδ T cell activator is a compound named (E)-5-hydroxy-3-methylpent-3-enyl pyrophosphonate, of Formula II:

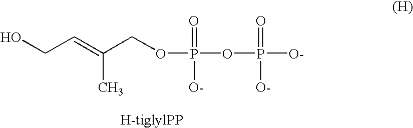

Specific examples of compounds also include: (E)1-pyrophosphonobuta-1,3-diene; (E)1-pyrophosphonopenta-1,3-diene; (E)1-pyrophosphono-4-methylpenta-1,3-diene; (E,E)1-pyrophosphono-4,8-dimethylnona-1,3,7-triene; (E,E,E)1-pyrophosphono-4,8,12-trimethyltrideca-1,3,7,11-tetraene; (E,E)1-triphosphono-4,8-dimethylnona-1,3,7-triene; 4-triphosphono-2-methylbutene; α,β-di[3-methylpent-3-enyl]-pyrophosphonate; 1-pyrophosphono-3-methylbut-2-ene; α,β-di-[3-methylbut-2-enyl]-triphosphonate; α,β-di-[3-methylbut-2-enyl]-pyrophosphonate; allyl-pyrophosphonate; allyl-triphosphonate; α,γ-di-allyl-pyrophosphonate; α,β-di-allyl-triphosphonate; (E,E)4-[(5′-pyrophosphono-6′-methyl-penta-2′,4′-dienyloxymethyl)-phenyl]-phenyl-methanone; (E,E)4-[(5′-triphosphono-6′-methyl-penta-2′,4′-dienyloxymethyl)-phenyl]-phenyl-methanone; (E,E,E)[4-(9′-pyrophosphono-2′,6′-dimethyl-nona-2′,6′,8′-trienyloxymethyl)-phenyl]-phenyl-methanone; (E,E,E)[4-(9′-pyrophosphono-2′,6′,8′-trimethyl-nona-2′,6′,8′-trienyloxymethyl)-phenyl]-phenyl-methanone; 5-pyrophosphono-2-methypentene; 5-triphosphono-2-methypentene; α,γ-di-[4-methylpent-4-enyl]-triphosphonate; 5-pyrophosphono-2-methypent-2-ene; 5-triphosphono-2-methypent-2-ene; 9-pyrophosphono-2,6-dimethynona-2,6-diene; 9-triphosphono-2,6-dimethynona-2,6-diene; α,γ-di-[4,8-dimethylnona-2,6-dienyl]-triphosphonate; 4-pyrophosphono-2-methylbutene; 4-methyl-2-oxa-pent-4-enyloxymethylpyrophosphate; 4-methyl-2-oxa-pent-4-enyloxymethyltriphosphate; α,β-di-[4-methyl-2-oxa-pent-4-enyloxymethyl]-pyrophosphate; and α,γ-di-[4-methyl-2-oxa-pent-4-enyloxymethyl]-triphosphate.

In other particular embodiments, the phosphoantigen can be selected from the group consisting of: 3-(halomethyl)-3-butanol-1-yl-diphosphate; 3-(halomethyl)-3-pentanol-1-yl-diphsophate; 4-(halomethyl)-4-pentanol-1-yl-diphosphate; 4-(halomethyl)-4-hexanol-1-yl-diphosphate; 5-(halomethyl)-5-hexanol-1-yl-diphosphate; 5-(halomethyl)-5-heptanol-1-yl-diphosphate; 6-(halomethyl)-6-heptanol-1-yl-diphosphate; 6-(halomethyl)-6-octanol-1-yl-diphosphate; 7-(halomethyl)-7-octanol-1-yl-diphosphate; 7-(halomethyl)-7-nonanol-1-yl-diphosphate; 8-(halomethyl)-8-nonanol-1-yl-diphosphate; 8-(halomethyl)-8-decanol-1-yl-diphosphate; 9-(halomethyl)-9-decanol-1-yl-diphosphate; 9-(halomethyl)-9-undecanol-1-yl-diphosphate; 10-(halomethyl)-10-undecanol-1-yl-diphosphate; 10-(halomethyl)-10-dodecanol-1-yl-diphosphate; 11-(halomethyl)-11-dodecanol-1-yl-diphosphate; 11-(halomethyl)-11-tridecanol-1-yl-diphosphate; 12-(halomethyl)-12-tridecanol-1-yl-diphosphate; 12-(halomethyl)-12-tetradecanol-1-yl-diphosphate; 13-(halomethyl)-13-tetradecanol-1-yl-diphosphate; 13-(halomethyl)-13-pentadecanol-1-yl-diphosphate; 14-(halomethyl)-14-pentadecanol-1-yl-diphosphate; 14-(halomethyl)-14-hexadecanol-1-yl-diphosphate; 15-(halomethyl)-15-hexadecanol-1-yl-diphosphate; 15-(halomethyl)-15-heptadecanol-1-yl-diphosphate; 16-(halomethyl)-16-heptadecanol-1-yl-diphosphate; 16-(halomethyl)-16-octadecanol-1-yl-diphosphate; 17-(halomethyl)-17-octadecanol-1-yl-diphosphate; 17-(halomethyl)-17-nonadecanol-1-yl-diphosphate; 18-(halomethyl)-18-nonadecanol-1-yl-diphosphate; 18-(halomethyl)-18-eicosanol-1-yl-diphosphate; 19-(halomethyl)-19-eicosanol-1-yl-diphosphate; 19-(halomethyl)-19-heneicosanol-1-yl-diphosphate; 20-(halomethyl)-20-heneicosanol-1-yl-diphosphate; 20-(halomethyl)-20-docosanol-1-yl-diphosphate; 21-(halomethyl)-21-docosanol-1-yl-diphosphate; and 21-(halomethyl)-21-tricosanol-1-yl-diphosphate.

More particularly, the phosphoantigen can be selected from the group consisting of: 3-(bromomethyl)-3-butanol-1-yl-diphosphate (BrHPP); 5-bromo-4-hydroxy-4-methylpentyl pyrophosphonate (CBrHPP); 3-(iodomethyl)-3-butanol-1-yl-diphosphate (IHPP); 3-(chloromethyl)-3-butanol-1-yl-diphosphate (CIHPP); 3-(bromomethyl)-3-butanol-1-yl-triphosphate (BrHPPP); 3-(iodomethyl)-3-butanol-1-yl-triphosphate (IHPPP); α,γ-di-[3-(bromomethyl)-3-butanol-1-yl]-triphosphate (diBrHTP); and α,γ-di[3-(iodomethyl)-3-butanol-1-yl]-triphosphate (diIHTP).

In another particular embodiment, the phosphoantigen can be selected from the group consisting of: 3,4-epoxy-3-methyl-1-butyl-diphosphate (Epox-PP); 3,4,-epoxy-3-methyl-1-butyl-triphosphate (Epox-PPP); α,γ-di-3,4,-epoxy-3-methyl-1-butyl-triphosphate (di-Epox-TP); 3,4-epoxy-3-ethyl-1-butyl-diphosphate; 4,5-epoxy-4-methyl-1-pentyl-diphosphate; 4,5-epoxy-4-ethyl-1-pentyl-diphosphate; 5,6-epoxy-5-methyl-1-hexyl-diphosphate; 5,6-epoxy-5-ethyl-1-hexyl-diphosphate; 6,7-epoxy-6-methyl-1-heptyl-diphosphate; 6,7-epoxy-6-ethyl-1-heptyl-diphosphate; 7,8-epoxy-7-methyl-1-octyl-diphosphate; 7,8-epoxy-7-ethyl-1-octyl-diphosphate; 8,9-epoxy-8-methyl-1-nonyl-diphosphate; 8,9-epoxy-8-ethyl-1-nonyl-diphosphate; 9,10-epoxy-9-methyl-1-decyl-diphosphate; 9,10-epoxy-9-ethyl-1-decyl-diphosphate; 10,11-epoxy-10-methyl-1-undecyl-diphosphate; 10,11-epoxy-10-ethyl-1-undecyl-diphosphate; 11,12-epoxy-11-methyl-1-dodecyl-diphosphate; 11,12-epoxy-11-ethyl-1-dodecyl-diphosphate; 12,13-epoxy-12-methyl-1-tridecyl-diphosphate; 12,13-epoxy-12-ethyl-1-tridecyl-diphosphate; 13,14-epoxy-13-methyl-1-tetradecyl-diphosphate; 13,14-epoxy-13-ethyl-1-tetradecyl-diphosphate; 14,15-epoxy-14-methyl-1-pentadecyl-diphosphate; 14,15-epoxy-14-ethyl-1-pentadecyl-diphosphate; 15,16-epoxy-15-methyl-1-hexadecyl-diphosphate; 15,16-epoxy-15-ethyl-1-hexadecyl-diphosphate; 16,17-epoxy-16-methyl-1-heptadecyl-diphosphate; 16,17-epoxy-16-ethyl-1-heptadecyl-diphosphate; 17,18-epoxy-17-methyl-1-octadecyl-diphosphate; 17,18-epoxy-17-ethyl-1-octadecyl-diphosphate; 18,19-epoxy-18-methyl-1-nonadecyl-diphosphate; 18,19-epoxy-18-ethyl-1-nonadecyl-diphosphate; 19,20-epoxy-19-methyl-1-eicosyl-diphosphate; 19,20-epoxy-19-ethyl-1-eicosyl-diphosphate; 20,21-epoxy-20-methyl-1-heneicosyl-diphosphate; 20,21-epoxy-20-ethyl-1-heneicosyl-diphosphate; 21,22-epoxy-21-methyl-1-docosyl-diphosphate; and 21,22-epoxy-21-ethyl-1-docosyl-diphosphate.

In a further particular embodiment, the phosphoantigen can be selected from the group consisting of: 3,4-epoxy-3-methyl-1-butyl-diphosphate (Epox-PP); 3,4,-epoxy-3-methyl-1-butyl-triphosphate (Epox-PPP); α,γ-di-3,4,-epoxy-3-methyl-1-butyl-triphosphate (di-Epox-TP); and uridine 5′-triphosphate-(3,4-epoxy methyl butyl) (Epox-UTP).

In another preferred embodiment, the phosphoantigen can be selected from the group consisting of: (E)-4-hydroxy-3-methyl-2-butenyl pyrophosphate (HDMAPP), (E)-5-hydroxy-4-methylpent-3-enyl pyrophosphonate (CHDMAPP) and (E)-5-hydroxy-3-methylpent-3-enyl pyrophosphonate (H-tiglylPP).

These compounds may be produced according to various techniques known per se in the art, some of which being disclosed in PCT Publications nos. WO 00/12516, WO 00/12519, WO 03/050128, WO 02/083720 and WO 03/009855, the disclosures of which are incorporated herein by reference.

In one preferred embodiment, the phosphoantigen is a γδ T cell activator and is a compound described in any one of PCT publication nos. WO 00/12516, WO 00/12519, WO 03/050128, WO 02/083720, WO 03/009855, WO 05/054258, WO 06/103 568 and WO 07/039,635, the disclosures of which Formulas and specific structures as well as synthesis methods are incorporated herein by reference. In another preferred embodiment, the phosphoantigen is a γδ T cell activator and is a compound selected from the group consisting of BrHPP, CBrHPP, EpoxPP, HDMAPP, CHDMAPP, NHDMAPP, H-angelylPP and H-tiglylPP.

Alternatively, although less potent in their functions as a γδ T cell activator, other activators for use in the present invention are phosphoantigens disclosed in WO 95/20673, isopentenyl pyrophosphate (IPP) (U.S. Pat. No. 5,639,653) and 3-methylbut-3-enyl pyrophosphonate (C—IPP). The disclosures of both references are incorporated herein by reference. Also encompassed are compounds that contain a phosphate moiety and act as γδ T cell inhibitors; one example is a compound disclosed in U.S. Pat. No. 6,624,151 B1, the disclosure of which is incorporated herein by reference.

Each of the foregoing references relating to compounds and their synthesis are incorporated herein by reference.

### Dosage of Preferred γδ T Cell Activators

As discussed, preferred compounds are selected which increase the biological activity of γδ T cells, preferably increasing the activation or proliferation of γδ T cells, particularly increasing cytokine secretion from γδ T cells or increasing the cytolytic activity of γδ T cells, with or without also stimulating the expansion of γδ T cells. For example, a γδ T cell activator allows the cytokine secretion by γδ T cells to be increased at least 2, 3, 4, 10, 50, 100-fold, as determined in vitro. Cytokine secretion and cytolytic activity can be assessed using any appropriate in vitro assay, or those described herein.

Optionally, in another aspect, the present invention relates to methods for the treatment of a disease, especially a proliferative disease, and more preferably a solid tumor, particularly a solid tumor having metastases, where a γδ T cell activator, especially a γδ T cell activator according to Formulas I to III. As used herein, the expression “Formulas Ito III”, designate all compounds derived from Formulas Ito III: I, II, IIa, III, Ma, IIIc 1, IIIa2, IIIa3, A, B, Mb, IIIb1, IIIb2, IIIb3, C, IIIc, IIIc1, IIIc2, IIIc3, D, E, F, G and H. Preferably, the γδ T cell activator selected from the group consisting of BrHPP, CBrHPP, EpoxPP, HDMAPP, CHDMAPP, NHDMAPP, H-angelylPP and H-tiglylPP, is administered in an amount and under conditions sufficient to stimulate the expansion of the γδ T cell population in a subject, particularly to reach 30-90% of total circulating lymphocytes, typically 40-90%, more preferably from 50-90%, conjointly with a chemotherapeutic agent. In typical embodiments, the conjoint therapy with a chemotherapeutic agent allows (e.g. does not prevent) the selective expansion of γδ T cells in a subject, to reach more than 10%, 20%, 30%, 40%, 50%, or up to 60-90% of total circulating lymphocytes. Percentage of total circulating lymphocytes can be determined according to methods known in the art. A preferred method for determining the percentage of γδ T cells in total circulating lymphocytes is by flow cytometry, examples of appropriate protocols described in the examples herein.

In another embodiment, the present invention relates to methods for the treatment of a disease, especially a proliferative disease, preferably a hematopoietic and a solid cancer, and more preferably a solid tumor, where a γδ T cell activator, especially a γδ T cell activator according to Formulas I to III, especially γδ T cell activator selected from the group consisting of BrHPP, CBrHPP, EpoxPP, HDMAPP, CHDMAPP, NHDMAPP, H-angelylPP and H-tiglylPP, is administered in an amount and under conditions sufficient to stimulate the expansion of the γδ T cell population in a subject, particularly to increase by more than 2-fold the number of γδ T cells in a subject, typically at least 4, 5 or 10-fold, more preferably at least 20-fold, conjointly with a IL-2 at a dose of about 7 to 9 MIU total daily in human, preferably about 8 MIU total in human daily. In typical embodiments, the conjoint administration with IL-2, at a dose of about 7 to 9 MIU total in human daily, preferably about 8 MIU total in human daily, allows the selective expansion of γδ T cells in a subject, to increase by at least 2, 4, 5, 10, 20, or 50-fold the number of γδ T cells in a subject, more preferably at least 100 fold. The number of γδ T cells in a subject is preferably assessed by obtaining a blood sample from a patient before and after administration of said γδ T cell activator and determining the difference in number of γδ T cells present in the sample. A preferred method for determining the number of γδ T cells by flow cytometry, examples of appropriate protocols described in the examples herein.

In another aspect, the present invention relates to methods for the treatment of a disease, especially a proliferative disease, and more preferably a solid tumor, particularly a solid tumor having metastases, where a γδ T cell activator, especially a γδ T cell activator according to Formulas I to III, especially γδ T cell activator selected from the group consisting of BrHPP, CBrHPP, EpoxPP, HDMAPP, CHDMAPP, NHDMAPP, H-angelylPP and H-tiglylPP, is administered in an amount and under conditions sufficient to stimulate the expansion of the γδ T cell population in a subject, particularly to reach a circulating γδ T cell count of at least 500 γδ T cells/mm³ in a subject, typically at least 1000 γδ T cells/mm³, more preferably at least 2000 γδ T cells/mm³, conjointly with interleukin-2 polypeptide.

The circulating γδ T cell count in a subject is preferably assessed by obtaining a blood sample from a patient before and after administration of said γδ T cell activator and determining the number of γδ T cells in a given volume of sample. A preferred method for determining the number of γδ T cells by flow cytometry, examples of appropriate protocols described in Example 1.

In a further aspect, the present invention relates to an in vivo regimen for the treatment of a proliferative disease, especially a solid tumor and more particularly a solid tumor having metastases, where a γδ T cell activator, especially a γδ T cell activator according to Formulas Ito III, especially γδ T cell activator selected from the group consisting of BrHPP, CBrHPP, EpoxPP, HDMAPP, CHDMAPP, NHDMAPP, H-angelylPP and H-tiglylPP, is administered to a warm-blooded animal, especially a human, in a dose that is higher (preferably at least 10%, 20%, 30% higher) than the single administration Efficient Concentration value giving half of the maximum effect (EC50) of γδ T cell biological activity or population expansion, or more preferably a dose that is at least 50%, or more preferably at least 60%, 75%, 85% or preferably between about 50% and 100% of the single administration Efficient Concentration value giving the maximum effect, conjointly with interleukin-2 polypeptide. In an embodiment, the γδ T cell activator is of Formula A, B or C is administered at a dose comprised between 2 and 60 mg/kg. In an embodiment, the γδ T cell activator is of Formula D, E, F G or H is administered at a dose comprised between 20 μg/kg and 2.5 mg/kg.

Preferably, dosage (preferably as a single administration at the beginning of a treatment cycle) of a γδ T cell activator compound of Formula Ito III for treatment is between about 1 μg/kg and about 1.2 g/kg. It will be appreciated that the above dosages related to a group of compounds, and that each particular compound may vary in optimal doses, as further described herein for exemplary compounds. Nevertheless, compounds are preferably administered in a dose sufficient to significantly increase the biological activity of γδ T cells or to significantly increase the γδ T cell population in a subject. Said dose is preferably administered to the human by intravenous (i.v.) administration during 2 to 180 min, preferably 2 to 120 min, more preferably during about 5 to about 60 min, or most preferably during about 30 min or during about 60 min. In preferred exemplary compounds, a compound of Formula II to III is administered in a dosage (single administration) between 1 μg/kg and about 1.2 g/kg, more preferably between about 1 μg/kg and about 100 mg/kg. In the framework of the present invention, the expression “Formulas II to III”, designate all compounds derived from Formulas II to III: II, IIIa, III, IIIa, IIIa1, IIIa2, IIIa3, A, B, Mb, IIIb1, IIIb2, IIIb3, C, IIIc, IIIc 1, IIIc2, IIIc3, D, E, F, G and H. Most preferably, dosage (single administration) for three-weekly or four-weekly treatment (treatment every three weeks or every third week) is between about 1 μg/kg and about 1.2 g/kg, preferably between about 1 μg/kg and about 100 mg/kg. In preferred exemplary compounds, a compound of Formula IIIa, especially a compound of Formula A or B is administered in a dosage (single administration) between about 1 mg/kg and about 100 mg/kg, preferably between about 10 mg/kg and about 80 mg/kg, more preferably between about 20 mg/kg and about 30 mg/kg. In preferred exemplary compounds, a compound of Formula Inc, especially, a compound of Formula D, E, F, G or H, is administered in a dosage (single administration) between about 1 μg/kg and about 100 mg/kg, preferably between about 10 μg/kg and about 20 mg/kg, more preferably between about 20 μg/kg and about 5 mg/kg, even more preferably between about 20 μg/kg and 2.5 mg/kg. Most preferably, dosage (single administration) for three-weekly or four-weekly treatment (treatment every three weeks or every third week) is between about 1 μg/kg and about 100 mg/kg, preferably between about 10 μg/kg and about 20 mg/kg, more preferably between about 20 μg/kg and about 5 mg/kg, even more preferably between about 20 μg/kg and 2.5 mg/kg. Further detail on dosages and administration and examples of dose response experiments using γδ T cell activator in mice and primate models are provided in PCT Application WO 04/050 096, the disclosure of which is incorporated herein by reference.

Unless otherwise indicated, the dosages for administration to a warm blooded animal, particularly humans provided herein are indicated in pure form (anionic form) of the respective compound. Purity level for the active ingredient depending on the synthesis batch can be used to adjust the dosage from actual to anionic form and vice-versa.

### Administration of γδ T Cell Activators of Formula I to III

It will be appreciated that the above dosages related to a group of compounds, and that each particular compound may vary in optimal doses, as further described herein for exemplary compounds. Nevertheless, compounds are preferably administered in a dose sufficient to significantly increase the biological activity of γδ T cells or to significantly increase the γδ T cell population in a subject. Said dose is preferably administered to the human by intravenous (i.v.) administration during 2 to 180 min, preferably 2 to 120 min, more preferably during about 5 to about 60 min, or most preferably during about 30 min or during about 60 min. As further described herein, the invention discloses that a brief stimulation of γδ T cell activity is sufficient to achieve the γδ T cell regulating effect. Thus, preferably where a γδ T cell activating compound has a short serum half-life, for example having a serum half-life of less than about 48 hours, less than about 24 hours, or less than about 12 hours, a rapid infusion is used. Said rapid infusion is preferably between about 10 minutes and 60 minutes, or more preferably about 30 minutes.

In another embodiment, said dose is preferably administered to the human by oral administration, in a solid or liquid dosage form, including but not limited to tablets, capsules, sachets, lozenges, powders, pills, or granules, and the solid dosage form can be, for example, a fast melt dosage form, chewable form, controlled release dosage form, lyophilized dosage form, delayed release dosage form, extended release dosage form, pulsatile release dosage form, mixed immediate release and controlled release dosage form, or a combination thereof.

### Interleukin-2

As indicated above, the method is based on the use of particular combinations of active agents, according to particular schedules. The invention more preferably uses a cytokine in combination with a γδ T cell activator, the cytokine being an interleukin-2 polypeptide.

The interleukin-2 polypeptide may be of human of animal origin, preferably of human origin. It may comprise the sequence of a wild-type human (or animal) IL-2 protein, or any biologically active fragment, variant or analogue thereof, i.e., any fragment, variant or analogue capable of binding to an IL-2 receptor and of inducing activation of γδ T cells in the method of this invention.

The sequence of reference, wild-type human interleukin-2 proteins is available in the art, such as in Genbank, under accession numbers NP000577; AAK26665; P01585; XP035511 for instance.

The term “variant” designates, in particular, any natural variants, such as those resulting from polymorphism(s), splicing(s), mutation(s), etc. Such naturally-occurring variants may thus comprise one or several mutation, deletion, substitution and/or addition of one or more amino acid residues, as compared to a reference IL-2 protein sequence.

The term “variant” also includes IL-2 polypeptides originating from various mammalian species, such as for instance rodent, bovine, porcine, equine, etc. More preferably, the IL-2 polypeptide is of human origin, i.e., comprises the sequence of a human EL-2 protein or a variant, fragment or analogue thereof.

The term “variant” also includes synthetic IL-2 variants, such as any synthetic polypeptide comprising one or several mutation, deletion, substitution and/or addition of one or more amino acid residues, as compared to a reference IL-2 protein sequence, and capable of binding to an IL-2 receptor and of inducing activation of γδ T cells in the method of this invention. Preferred synthetic IL-2 variants have at least 75% identity in amino acid sequence with the primary sequence of an IL-2 reference protein, more preferably at least 80%, even more preferably at least 85 or 90%. The identity between sequences may be determined according to various known methods such as, typically, using the CLUSTAL method.

Variants also include IL-2 polypeptides encoded by a nucleic acid sequence that hybridize, under conventional, moderate stringency, with the nucleic acid sequence encoding a reference IL-2 protein, or a fragment thereof. Hybridization conditions are, for instance incubation at 40-42° C. for 12 hours in 50% formamide, 5×SSPE, 5×Denhardt's solution, 0.1% SDS.

The IL-2 polypeptide may also be any fragment of a reference IL-2 protein which retains the ability to bind to an IL-2 receptor and to induce activation of γδ T cells in the method of this invention. Such fragments contain, at least, one functional domain of IL-2, such as the receptor binding site. Fragments contain preferably at least 40%, 50% or, preferably, at least 60% of the complete reference sequence.

Analogues designate polypeptides using the same receptor as Interleukin-2 and thus mediating similar activation signal in a γδ T lymphocyte.

The interleukin-2 polypeptide may further comprise heterologous residues added to the natural sequence, such as additional amino acids, sugar, lipids, etc. This may also be chemical, enzymatic or marker (e.g., radioactive) groups. The added residues or moiety may represent a stabilizing agent, a transfection-facilitating agent, etc.

The IL-2 polypeptides may be in soluble, purified form, or conjugated or complexed with an other molecule, such as a biologically active peptide, protein, lipid, etc. The IL-2 polypeptide may be produced according to techniques known in the art, such as by chemical synthesis, enzymatic synthesis, genetic (e.g., recombinant DNA) synthesis, or a combination thereof. An IL-2 polypeptide of pharmaceutical grade may also be obtained from commercial sources.

The interleukin-2 used in the Examples herein is Proleukin™ (aldesleukin). Proleukin™, which is described in its product information leaflet as follows: a human recombinant interleukin-2 product, is a highly purified protein with a molecular weight of approximately 15 300 daltons. The chemical name is des-alanyl-1, serine-125-human interleukin-2. Proleukin™ is a lymphokine, produced by recombinant DNA technology using a genetically engineered E. Coli strain containing an analog to the human interleukin-2 gene. Genetic engineering techniques were used to modify the human IL-2 gene, and the resulting expression clone encodes a modified human interleukin-2. The recombinant forms differs from native interleukin-2 in the following ways: a) Proleukin™ is not glycosylated because it is derived from E. Coli; b) the molecule has no N-terminal alanine; the codon for this amino acid was deleted during the genetic engineering procedure; c) the molecule has serine substituted for cysteine at amino acid position 125; this was accomplished by site specific manipulation during the genetic engineering procedure; and d) the aggregation state of Proleukin™ is likely to be different from that of native interleukin-2. Proleukin™ biological potency is determined by a lymphocyte proliferation assay and is expressed in international units (IU) as established by the World Health Organization I international strand for interleukin-2 (human). The relationship between potency and protein mass is as follows: 18 MIU Proleukin™=1.1 mg protein.

The dose of IL-2 to be administered is generally expressed per square meter. The body surface of a human subject lies between 1.5 to 2.1 m² depending on the size and weight of the subject. The body surface area (BSA) can be determined using different Formulas such as the Mosteller Formula or the Dubois and Dubois Formula.

BSA(m²)=([Height(cm)×Weight(kg)]/3600)1/2 The Mosteller Formula

BSA(m²)=0.20247×Height(m)^{0.725}×Weight(kg)^{0.425} The DuBois and DuBois² Formula

The average body surface is of about 1.7 m².

The IL-2 polypeptide is preferably administered by injections of between 7 and 9 MIU total in human daily, over a period of 1 to 10 days. Preferably, daily doses of 7.5 to 8.5 MIU total in human daily, most preferably doses of 8 MIU total in human daily are being administered. With respect to the body surface area equivalence and the variability, the IL-2 polypeptide can be administered for example by injections of between 3.3 and 6 MIU/m² daily, over a period of 1 to 10 days. Preferably, daily doses from 3.5 to 5.5 MIU/m² are being administered.

The preferred dose for IL-2, expressed in international units, can be transposed to any available or commercial interleukin-2. In the present inventions, the inventors have used the commercial interleukin-2 polypeptide Proleukin™ by Chiron. It will be appreciated that any other interleukin-2 may be used, as long as the quantity of product to be administered is adjusted according to the dosage of the IL-2 chosen. Such dosage is easily carried on by the skilled artisan using standard analytical methods (see Hammerling et al, J. Pharm. And Biomed. Anal 1992, p. 547-553).

### Treatment Cycles

Treatment cycles can be carried out in a number of ways. Generally, a cycle comprises at least one administration of a γδ T cell activator and the administration of IL-2.

The IL-2 treatment is preferably maintained over between 1 and 9 days, even more preferably during 3 to 7 days (e.g. for 3 days, 4 days, 5 days, 6 days or 7 days), optionally where said days are consecutive. Preferably, the IL-2 treatment commences within 24 hours (preferably on the same day) of the administration of γδ T cell activator. Optimum effect seems to be achieved after 5 consecutive days of treatment with IL-2.

In a preferred embodiment, the active ingredients are administered through different schedules: the γδ T cell activator is administered as a single shot, at the beginning of the treatment cycle, and the interleukin-2 polypeptide is administered over a prolonged period of time, typically between 1 and 9 days. As shown in the experimental section, such administration schedule provides a remarkable increase in the activity of γδ T cells in a subject

Most preferably, the γδ T cell activator will be administered on the day that IL-2 treatment commences, and the IL-2 treatment is administered on at least 5 consecutive days. The γδ T cell activator is typically administered only on a single day (e.g. is not re-administered during the subsequent days of IL-2 treatment).

It will be appreciated that the daily dose of IL-2 and/or γδ T cell activator may be administered as a single injection or in several times, for example in two equal injections per day.

Optimum results appear to be achieved when the γδ T cell activator is administered on a single day at the beginning of each treatment cycle.

Preferably, if multiple administrations of the γδ T cell activator are provided, the administrations that give rise to a γδ T cell proliferation are separated by a period of time sufficient to prevent “exhaustion” of the γδ T cells. Exhaustion can be characterized by reduction in ability to produce cytokines or to proliferate in response to the γδ T cell activator, in comparison to that observed when the γδ T cells are treated with a first dose or a preceding dose of the γδ T cell activator. Generally, the γδ T cell rate (number of γδ T cells), is allowed to return to substantially basal rate prior to a second administration of the γδ T cell activator compound. At least about one week, but more preferably at least about two weeks, or up to eight weeks are required for a patient's γδ T cell rate to return to substantially basal rate.

In another aspect, the γδ T cell activator is administered in multiple doses, the administration of successive doses of the γδ T cell activator takes place at the beginning of a treatment cycles, which are separated by at least 2, 3 or 4 6, 8, 12, 24, or 36 weeks. In preferred aspects, one first dose of a γδ T cell activator is administered, and one or more (preferably at least two) further doses of γδ T cell activator are administered in different treatment cycles.

In a preferred embodiment, a treatment cycle will be defined as described hereafter: On day 1, the γδ T cell activator is administered to the patient. Preferably in a short time after the γδ T cell activator administration, typically less than one hour after, IL-2 is administered to the patient. On following days (day 2, 3, 4, and 5), IL-2 is administered to the patient. Following these administrations, the γδ T cell population is activated and the patient is allowed to rest until the γδ T cell count has returned to basal rate, typically around day 21. This treatment scheme represents a treatment cycle A as shown in FIG. 4. Preferably, the treatment cycle A will be repeated at least 2 times, more preferably, the treatment cycle will be repeated three times.

Thus, the course of a preferred treatment cycle with a γδ T cell activator is an at least 1-weekly cycle, but more preferably at least a 2-weekly cycle (at least about 14 days), or more preferably at least 3-weekly or 4-weekly, though cycles anywhere between 2-weekly and 4-weekly are preferred. Also effective and contemplated are cycles of up to 8-weekly, for example 5-weekly, 6-weekly, 7-weekly or 8-weekly.

A subject will preferably be treated for at least two cycles, or more preferably for at least three cycles. In other aspect, treatment may continue for a greater number of cycles, for example at least 4, 5, 6 or more cycles can be envisioned. At the end of each cycle, the cycle of dosing may be repeated for as long as clinically tolerated and the tumor is under control or until tumor regression. Tumor “control” is a well recognized clinical parameter, as defined above. In a preferred embodiment, the cycle of dosing is repeated for up to about eight cycles.

The active ingredients may be administered through different routes, typically by injection or oral administration. Injection may be carried out into various tissues, such as by intravenous, intra-peritoneal, intra-arterial, intra-muscular, intra-dermic, subcutaneous, etc. Preferred administration routes for the γδ T cell activators are intravenous, intra-muscular or oral. Preferred administration routes for the cytokine are subcutaneous, intravenous and intra-muscular.

Further aspects and advantages of the present invention will be disclosed in the experimental section, which should be regarded as illustrative and not limiting the scope of the present application.

Combination with Other Therapeutic Treatments

According to another embodiment, the compositions according to the invention may further comprise another therapeutic agent, including agents normally utilized for the particular therapeutic purpose for which the γδ T cell activator is being administered. The additional therapeutic agent will normally be present in the composition in amounts typically used for that agent in a monotherapy for the particular disease or condition being treated. Such therapeutic agents include, but are not limited to, therapeutic agents used in the treatment of cancers, therapeutic agents used to treat infectious disease, therapeutic agents used in other immunotherapies, tyrosine kinase inhibitors, chemotherapies, cytokines (such as IL-15), antibodies (such as for example rituximab, bezacuzimab) and fragments thereof.

When one or more agents are used in combination in a therapeutic regimen, there is no requirement for the combined results to be additive of the effects observed when each treatment is conducted separately. Although at least additive effects are generally desirable, any increased effect, for example an anti-cancer effect, above one of the single therapies would be of benefit. Also, there is no particular requirement for the combined treatment to exhibit synergistic effects, although this is certainly possible and advantageous.

### Antibodies

The term “antibody,” as used herein, refers to polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids that is primarily responsible for antigen recognition. The terms variable light chain (V_{L}) and variable heavy chain (V_{H}) refer to these light and heavy chains respectively. The heavy-chain constant domains that correspond to the different classes of immunoglobulins are termed “alpha,” “delta,” “epsilon,” “gamma” and “mu,” respectively. The subunit structures and three-dimensional configurations of different classes of immunoglobulins are well known. IgG and/or IgM are the preferred classes of antibodies employed in this invention, with IgG being particularly preferred, because they are the most common antibodies in the physiological situation, because they are most easily made in a laboratory setting, and because IgGs are specifically recognized by Fc gamma receptors. Preferably the antibody of this invention is a monoclonal antibody. Particularly preferred are humanized, chimeric, human, or otherwise-human-suitable antibodies.

Within the context of this invention, the term “therapeutic antibody or antibodies” designates more specifically any antibody that functions to target cells in a patient and optionally to deplete targeted cells in a patient. In particular, therapeutic antibodies specifically bind to antigens present on the surface of the target cells, e.g. tumor specific antigens present predominantly or exclusively on tumor cells. Preferably, therapeutic antibodies include human Fc portions, or are capable of interacting with human Fc receptors. Therapeutic antibodies can target cells by any means, e.g. ADCC or otherwise, and can be “naked,” i.e. with no conjugated moieties, or they can be conjugated with compounds such as radioactive labels or toxins.

Typical examples of therapeutic antibodies of this invention are rituximab (Anti-CD20 antibody, MabThera®, Rituxan®), alemtuzumab and trastuzumab. Such antibodies may be used according to clinical protocols that have been authorized for use in human subjects. Additional specific examples of therapeutic antibodies include, for instance, epratuzumab, basiliximab, daclizumab, cetuximab, labetuzumab, sevirumab, tuvurimab, palivizumab, infliximab, omalizumab, efalizumab, natalizumab, clenoliximab, etc. Other examples of preferred therapeutic antibodies for use in accordance with the invention include anti-ferritin antibodies (US Patent Publication no. 2002/0106324), anti-p140 and anti-sc5 antibodies (WO 02/50122), the disclosures of each of the above reference being incorporated herein by reference. It will be appreciated that any antibody that can target cells, and optionally deplete the targeted cell, e.g. by ADCC, can benefit from the present methods. The efficient amount of therapeutic antibodies administered to the subject can be between about 0.1 mg/kg and about 20 mg/kg. The efficient amount of antibody depends however of the form of the antibody (whole Ig, or fragments), affinity of the mAb and pharmacokinetics parameter that must be determined for each particular antibodies.

In a preferred embodiment, the antibody is the rituximab. In a more particular embodiment, said antibody is administered at a dosage of less than 375 mg/m² per week. In another embodiment, the antibody is Campath. In a more particular embodiment, the antibody is Campath, and the antibody is administered at a dosage of less than 90 mg per week.

### Tyrosine Kinase Inhibitors

New blood vessel formation (angiogenesis) is a fundamental event in the process of tumor growth and metastatic dissemination. The vascular endothelial growth factor (VEGF) pathway is well established as one of the key regulators of this process. The VEGF/VEGF-receptor axis is composed of multiple ligands and receptors with overlapping and distinct ligand-receptor binding specificities, cell-type expression, and function. Activation of the VEGF-receptor pathway triggers a network of signaling processes that promote endothelial cell growth, migration, and survival from pre-existing vasculature. Particularly preferred anti-angiogenic agents inhibit signaling by a receptor tyrosine kinase including but not limited to VEGFR1, VEGFR-2,3 PDGFR-beta, Flt-3, c-Kit, p38 alpha and FGFR-1.

Examples of tyrosine kinase inhibitor (TKI) can be, but are not limited to: PTK-787 (Vatalanib, Novartis); SU11248 (Sunitimib, Pfizer), Sorafenib (Bayer/Onyx), AMG706 (Amgen), ZD1839 (gefitinib, Iressa, AstraZeneca), canertinib (Pfizer), OSI-774 (Erlotininb, Tarceva, OSI Pharmaceuticals), lapatinib (Tykerb, GlaxoSmithKline), dasatinib (Bristol-Myers Squibb), lestaurinib (Cephalon), nilotinib (AMN107, Novartis), imitanib (Glivec™, Gleevec™, Novartis).

### Chemotherapeutic Agents

Chemotherapeutic compounds or methods for use in accordance with the invention include alkylating agents, cytotoxic antibiotics such as topoisomerase I inhibitors, topoisomerase II inhibitors, plant derivatives, RNA/DNA antimetabolites, and antimitotic agents. Preferred examples may include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, taxol, gemcitabine, navelbine, transplatinum, 5-fluorouracil, vincristin, vinblastin and methotrexate, or any analog or derivative variant of the foregoing.

### Detailed Description of Preferred Treatment Regimens

### Renal Cell Carconima (RCC)

In specific embodiments, patients with renal cell carcinoma are administered a prophylactically or therapeutically effective amount of a γδ T cell activator in combination with the administration of a prophylactically or therapeutically effective amount of one or more other therapies useful for renal cell carcinoma treatment or management including but not limited to: an anti-angiogenic therapy or an HDAC inhibitor, most preferably a receptor tyrosine kinase inhibitor, an inhibitor or ras or raf kinase, or an antibody which specifically binds an angiogenic factor (e.g. VEGF). Most preferably the receptor tyrosine kinase inhibitor is an agent selected from the group consisting of: SU011248 (sunitinib), PTK787 and BAY 43-9006 (sorafenib). The most common subtype of RCC, accounting for 60-70% of cases, comprises clear or conventional cell carcinoma and is characterized by frequent inactivation of the Von Hippel-Lindau (VIM) tumor suppressor gene. RCC is generally considered resistant to conventional cytotoxic drugs.

### Breast Cancer

In specific embodiments, patients with breast cancer are administered a prophylactically or therapeutically effective amount of a γδ T cell activator in combination with a prophylactically or therapeutically effective amount of one or more other therapies useful for breast cancer treatment or management including, but not limited to: doxorubicin, epirubicin, the combination of doxorubicin and cyclophosphamide (AC), the combination of cyclophosphamide, doxorubicin and 5-fluorouracil (CAF), the combination of cyclophosphamide, epirubicin and 5-fluorouracil (CEF), tamoxifen, or the combination of tamoxifen and cytotoxic chemotherapy, an anti-angiogenic therapy or an HDAC inhibitor alone or in combination with any of the preceding. In certain embodiments, patients with metastatic breast cancer are administered a prophylactically or therapeutically effective amount of a γδ T cell activator in combination with the administration of a prophylactically or therapeutically effective amount of taxanes such as docetaxel and paclitaxel. In other embodiments, a patients with node-positive, localized breast cancer are administered a prophylactically or therapeutically effective amount of a γδ T cell activator in combination with the administration of a prophylactically or therapeutically effective amount of taxanes plus standard doxorubicin and cyclophosphamide for adjuvant treatment of node-positive, localized breast cancer.

### Colon Cancer

In specific embodiments, patients with colon cancer are administered a prophylactically or therapeutically effective amount of a γδ T cell activator in combination with the administration of a prophylactically or therapeutically effective amount of one or more other therapies useful for colon cancer treatment or management including but not limited to: the combination of 5-FU and leucovorin, the combination of 5-FU and levamisole, irinotecan (CPT-11) or the combination of irinotecan, 5-FU and leucovorin (IFL), oxaliplatin, or an anti-angiogenic therapy or an HDAC inhibitor alone or in combination with any of the preceding.

### Prostate Cancer

In specific embodiments, patients with prostate cancer are administered a prophylactically or therapeutically effective amount of a γδ T cell activator in combination with the administration of a prophylactically or therapeutically effective amount of one or more other therapies useful for prostate cancer treatment or management including but not limited to: external-beam radiation therapy, interstitial implantation of radioisotopes (i.e., palladium, and Iridium), chemotherapy regimens reported to produce subjective improvement in symptoms and reduction in PSA level including docetaxel, paclitaxel, estramustine/docetaxel, estramustine/etoposide, estramustine/vinblastine, and estramustine/paclitaxel, or an anti-angiogenic therapy or an HDAC inhibitor, alone or in combination with any of the preceding.

### Melanoma

In specific embodiments, patients with melanoma are administered a prophylactically or therapeutically effective amount of a γδ T cell activator in combination with the administration of a prophylactically or therapeutically effective amount of one or more other therapies useful for melanoma cancer treatment or management including but not limited to: dacarbazine (DTIC), nitrosoureas such as carmustine (BCNU) and lomustine (CCNU), agents with modest single agent activity including vinca alkaloids, platinum compounds, and taxanes, the Dartmouth regimen (cisplatin, BCNU, and DTIC), or an anti-angiogenic therapy or an HDAC inhibitor, alone or in combination with any of the preceding.

### Ovarian Cancer

In specific embodiments, patients with ovarian cancer are administered a prophylactically or therapeutically effective amount of a γδ T cell activator in combination with a prophylactically or therapeutically effective amount of one or more other therapies useful for ovarian cancer treatment or management including, but not limited to: intraperitoneal radiation therapy, total abdominal and pelvic radiation therapy, cisplatin, oxaliplatin, the combination of paclitaxel (Taxol) or docetaxel (Taxotere) and cisplatin or carboplatin, the combination of cyclophosphamide and cisplatin, the combination of cyclophosphamide and carboplatin, the combination of 5-fluorouracil (5-FU) and leucovorin, etoposide, liposomal doxorubicin, gerucitabine or topotecan, or an anti-angiogenic therapy or an HDAC inhibitor alone or in combination with any of the preceding. In a particular embodiment, patients with ovarian cancer that is platinum-refractory are administered a prophylactically or therapeutically effective amount of a γδ T cell activator in combination with the administration of a prophylactically or therapeutically effective amount of Taxol. The invention encompasses the treatment of patients with refractory ovarian cancer including administration of ifosfamide in patients with disease that is platinum-refractory, hexamethylmelamine (HAM) as salvage chemotherapy after failure of cisplatin-based combination regimens, and tamoxifen in patients with detectable levels of cytoplasmic estrogen receptor on their tumors.

### Lung Cancers

In specific embodiments, patients with non-small cell lung cancer and small lung cell cancer are administered a prophylactically or therapeutically effective amount of a γδ T cell activator in combination with the administration of a prophylactically or therapeutically effective amount of one or more other therapies useful for lung cancer treatment or management including but not limited to: thoracic radiation therapy, cisplatin, vincristine, doxorubicin, and etoposide, alone or in combination, the combination of cyclophosphamide, doxorubicin, vincristine/etoposide, and cisplatin (CAV/EP), or an anti-angiogenic therapy or an HDAC inhibitor alone or in combination with any of the preceding. In other specific embodiments, patients with non-small lung cell cancer are administered a prophylactically or therapeutically effective amount of a γδ T cell activator in combination with the administration of a prophylactically or therapeutically effective amount of one or more other therapies useful for lung cancer treatment or management including but not limited to: palliative radiation therapy, the combination of cisplatin, vinblastine and mitomycin, the combination of cisplatin and vinorelbine, paclitaxel, docetaxel or gemcitabine, the combination of carboplatin and paclitaxel, or an anti-angiogenic therapy or an HDAC inhibitor alone or in combination with any of the preceding.

### Non Hodgkin Lymphoma

In specific embodiments, patients with NHL are administered a prophylactically or therapeutically effective amount of a γδ T cell activator in combination with the administration of a prophylactically or therapeutically effective amount of one or more other therapies useful for NHL treatment or management including but not limited to chemotherapeutic agents such as cyclophosphamide, doxorubicin, vincristine, corticosteroids, but also monoclonal antibodies, such as rituximab (Rituxan™, Roche), most preferably, rituximab is administered.

The following examples will serve to further illustrate the present invention without, at the same time, however, constituting any limitation thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

### Example 1

### Flow Cytometry Detection of γδ T Cell Amplification

Blood samples (4 ml) were withdrawn into EDTA containing tubes. Tubes were shipped overnight at room temperature (RT) before flow cytometry analyses.

Peripheral lymphocytes were analyzed by flow cytometry on total blood, after triple staining with anti-Vδ2FITC, anti-CD3PE and anti-CD25PC5 antibodies (Vδ2-FITC: IMMU389 clone, Immunotech-Beckman-Coulter, Marseilles, France; CD3-PE: UCHT1 clone, Immunotech-Beckman-Coulter; CD25PC5: M-A251 clone, Becton Dickinson, Le Pont de Claix, France).

Briefly, 100 μl patient blood was incubated 15 min at RT with 10 μl anti-Vdelta2-FITC, 10 μl anti-CD3-PE and 10 μl anti-CD25PC5 antibodies. Antibodies were washed with 3 ml 1×PBS, centrifuged for 4 min at 1300 rpm at RT and supernatant was discarded. Red cells were lysed with the OptiLyse B reagent (Immunotech-Beckman-Coulter, Marseilles, France) according to the manufacturer's instructions. At the final step, stained white blood cells were recovered by centrifugation and resuspended in 300 μl 1×PBS+0.2% PFA. Flow cytometry analysis on viable cells was performed on a FacsSCAN apparatus (Becton Dickinson) with the FacsDiva software.

### Example 2

### Pharmacodynamic Characterization (Vγ9T Cell Expansion) of a Dose Range of IL-2 with a Fixed Dose of Phosphostim™ 200 Treatment in the Cynomolgus Macaque

### Materials and Methods

### Pharmacological Effect on Target Cells:

Follow up of Vγ9Vδ2 T cells in vivo in the macaque has been performed as usual by flow cytometry on days 0 (pre-dose) and 5 of treatment.

The staining of 24-hour whole blood samples is performed with the following antibody combination: for 50 μl of whole blood CD3-FITC (10 μl)/CCR5-PE (100)/Vγ9-PC5 (5 μl)/CD69-PC7 (5 μl).

### Reagents:

IL2: recombinant human IL2, Proleukin™, CHIRON; administered daily by subcutaneous (s.c.) route.

Phosphostim™ dose 48 mg/kg administered as 30-min infusion by intravenous (i.v.) route.

### Animals:

Twelve cynomolgus monkeys (M. fascicularis) aged 2.5 to 3.5 years at the start of the study. These animals were naïve of any previous treatment with phosphoantigens or IL2.

### Treatments:

The animals have been allocated to 4 dose-groups (0.075, 0.3, 1.2 and 2.4 million UI per day for 7 days), with 3 animals per dose, receiving IL2 daily for 7 days have, and a fixed dose of Phosphostim™ 200 (48 mg/kg) once, on the first day of treatment, with 3 animals per dose.

### IL2 Dose-Range to be Tested, with Correspondence

| | | Approximate Human |
|---|---|---|
| | Primate dose in | daily dose in |
| | million IU/animal (body | million IU/patient |
| Dose in million IU/m² | surface ~0.3 m²) | (body surface ~2 m²) |
| 0.25 | 0.075 | 0.50 |
| 1 | 0.3 | 2 |
| 4 | 1.2 | 8 |
| 8 | 2.4 | 16 |

### Results

Vγ9Vδ2 cell expansion is related to IL2 concentration after activation with a fixed dose (48 mg/kg) of Phosphostim™. Indeed, an IL2 dose range effect is observed in association with Phosphostim™ treatment on γδT cell expansion.
a. 0.075 MIU/animal/day (equivalent to 0.25 MIU/m² daily in human) IL2 dose is not sufficient to induce a γδT cell specific amplification.
b. 0.3 MIU/animal/day (equivalent to 1 MIU/m² daily in human) is the first efficient dose to allow a specific and significant γδcell expansion.
c. For further doses (1.2 and 2.4 MIU/animal/day, equivalent to 4 and 8 MIU/m² daily in human) the expansion is higher and the circulating V62 cells number has leveled off.

Monitoring of γδT cell expansion showed no effect in the 0.075 MIU/animal/daily IL-2 group, a weak γδT cell increase after 0.3 MIU animal/daily IL-2 co-treatment and a significantly higher increase after 1.2 and 2.4 MIU/animal/daily, suggesting the maximal response was already reached at 1.2 MIU/animal/daily (see FIG. 1). The most efficient dose is 1.2 MIU/animal/day (equivalent to 4 MIU/m² daily in human) as a further increase of IL-2 does not lead to an increased expansion.

The results of this study highlight that the dose inducing the maximum effect, reaching an expansion plateau of γδ T cells is of about 4 MIU/m² daily. Similar results were produced in a human clinical trial (as detailed in Example 3).

### Example 3

### IL-2 Dose Effect Comparison In Vivo in Phase IIa in mRCC

A phase II clinical trial in mRCC was set up, to test the efficacy of a therapeutic regimen with a fixed dose of Phosphostim™ (750 mg/m², 50 ml infusion in 30 min) and two doses of IL-2.

Patients in Arm A were treated with an IL-2 dose of 2 MIU total daily (equivalent to approximately 1 MIU/m² daily). Patients in Arm B were treated with an IL-2 dose of 8 MIU total daily. For practical reasons, and as a human body surface varies from 1.5 to 2.1 m² depending on each variable of the patient, a single dose for all the patients of Arm B was fixed. It will be appreciated that when taking the variations in body surface into account, this 8 MIU total daily dose of IL-2 can also be expressed as a range from 3.3 to 6 MIU/m² daily.

Patients suffering from mRCC were treated with a fixed dose of Phosphostim™ (750 mg/m², i.v. infusion of 30 min) and IL-2, at a dose of 2 MIU subcutaneous (s.c.) daily (arm A, 18 patients), or 8 MIU s.c. daily (arm B, 21 patients).

Phosphostim™ was administered once on day 1 which is the first day of treatment. IL-2 is administered daily s.c. from day 1 to day 5. On day 1, IL-2 is administered 10 minutes after the end of Phosphostim™ infusion.

γδ T cell population was measured through cytometry on day 0 (before the beginning of the treatment) and on day 7. The expansion of γδ T cell was obtained in fold increase of expansion compared to the basal γδ T cell count.

The 21 patients treated with 8 MIU total daily (equivalent to 3.3 to 6 MIU/m² daily) showed a significantly better γδ T cell expansion than the 18 patients treated with 2 MIU total daily (equivalent to 0.95 to 1.3 MIU/m² daily).

All patients showed a good tolerance to the doses of IL-2 administered, especially, none of the patients in arm B experienced an adverse toxicity effect (data not shown).

A specific pharmacological expansion of γδ T cell with Phosphostim™ was observed, and this expansion was related to dose of IL-2 administered. The inventors have found out that at a dose of 8 MIU total in human daily, the γδT cell expansion is significantly higher. Moreover, despite an increased dose of IL-2 compared to previous publications, this expansion is still very specific to γδ T cells. This result is highlighted in FIG. 3, where other cell subsets, such as NK and global T cells, are not expanded with both the doses of IL-2 of 2 MIU and 8 MIU total daily.

### Example 43

### IL-2 Dose Effect Comparison In Vivo in Phase II in NHL

This example shows the effect of the IL-2 dose on γδ T cell subset on the first results obtained in another clinical trial. 5 patients suffering from B-NHL are treated with 4 MIU total in human daily (arm A, 3 patients), or 8 MIU total in human daily (arm B, 2 patients). The treatment regimen used for this study is set out in FIG. 4B.

Rituxan™ (rituximab) is administered at a dose of 375 mg/m², on day 0 and then on day 7, 14 and 21, which corresponds to the common Rituxan™ treatment. Phosphostim™ is administered once on day 7 which is the first day of treatment. IL-2 is administered daily subcutaneous (s.c.) from day 7 to day 11. On day 7, IL-2 is administered 10 minutes after the end of Phosphostim™ infusion.

γδ T cell population is measured through cytometry on day 0 (before the beginning of the treatment) and on day 145 (corresponds to day 7 from the beginning of the Phosphostim™ treatment. The expansion of γδ T cell is obtained in fold increase compared to the basal γδ T cell count.

The 2 patients treated with 8 MIU total in human daily (equivalent to approximately 4 MIU/m² daily) show a significantly better γδ T cell expansion (mean of 46 fold increase) than the 3 patients treated with 4 MIU total in human daily (mean of 17 fold increase).

All patients showed a good tolerance to the high dose of IL-2 administered (in Arm B), especially, there was no significant difference in toxicity in Arm B compared to Arm A.

The results (FIGS. 6 and 7) highlight that the patients treated with a higher dose of IL-2 (8 MIU total daily) shows a much better γδ T cell amplification than patients treated with a lower dose of IL-2. The graph also shows that the quantity of BrHPP only as a slight influence on the γδ T cell amplification compared to the effect of the IL-2 dose.

1-68. (canceled)

69. A method of regulating the activity of γδ T cells or killing a tumor cell in a mammalian subject comprising conjointly administering to a subject an effective amount of a yd T cell activator and an interleukin-2 polypeptide, the interleukin-2 polypeptide being administered at a dose of between 3.3 and 6 MIU/m² daily.

70. The method of claim 69, wherein the interleukin-2 polypeptide being administered at a dose of between 7 and 9 MIU total daily.

71. The method of claim 69, wherein said γδ T cell activator is administered as a single dose on the first day of a treatment cycle with said γδ T cell activator.

72. The method of claim 69, wherein said interleukin-2 is administered:
a) on at least two days during the first 10 days of said treatment cycle;
b) on at least three days during the first 10 days of a treatment cycle;
c) on at least five days during the first 10 days of a treatment cycle;
d) on at least 3 consecutive days of a treatment cycle;
e) on at least 5 consecutive days of a treatment cycle;
f) on at least day 1 to day 3 of a treatment cycle; or
g) on at least day 1 to day 5 of a treatment cycle.

73. The method of claim 69 comprising:
a) administering to the subject an effective amount of a γδ T cell activator on a first day, and
b) administering to the subject an interleukin-2 polypeptide on at least 3 days within the days starting of said administration of a γδ T activator, wherein the interleukin-2 is administered at a dose of between 3.3 to 6 MIU/m² daily or at a dose of between 7 and 9 MIU total daily in human.

74. The method according to claim 69, wherein said interleukin-2 polypeptide is aldesleukin.

75. The method according to claim 69, wherein the γδ T cell activator is:
a) of the Formula Ito III; or
b) of the Formula A, B, C, D E, F, G or H.

76. The method according to claim 69, wherein the dose of γδ T cell activator to be administered is comprised between 1 μg/kg to 100 mg/kg.

77. The method according to claim 69, wherein the tumor to be treated is a solid tumor, a lymphoma or leukemia.

78. The method according to claim 69, further comprising conjointly administering a chemotherapeutic agent to said subject.

79. The method according to claim 78, wherein said chemotherapeutic agent is selected from the group consisting of antibodies, preferably rituximab, or tyrosine kinase inhibitors, preferably imatinib, sunitinib or sorafenib.

80. A product comprising a γδ T cell activator and an interleukin-2 polypeptide, for separate use, for regulating the activity of γδ T cells in a mammalian subject, wherein the dose of IL-2 is between 3.3 to 6 MIU/m² daily.

81. The product of claim 80, wherein the dose of IL-2 is between 7 and 9 total daily.

82. The product of claim 80, wherein said interleukin-2 polypeptide is aldesleukin.

83. The product of claim 80, wherein the γδ T cell activator is:
a) of the Formula Ito III; or
b) of the Formula A, B, C, D E, F, G or H.

84. The product of claim 83, wherein the dose of γδ T cell activator to be administered is comprised between 1 μg/kg and 100 mg/kg.

85. The product of claim 83, wherein the mammalian is a human subject suffering from a cancer or an infectious disease.

86. The product of claim 83, wherein the human subject suffers from a cancer selected from a solid tumor cancer or a hematopoietic cancer.

87. The product of claim 83, wherein a further chemotherapeutic agent is administered conjointly with the γδ T cell activator and the IL-2 polypeptide.

88. The product of claim 87, wherein said chemotherapeutic agent is selected from the group consisting of antibodies, preferably rituximab, or tyrosine kinase inhibitors, preferably imatinib, sunitinib or sorafenib.
